(19) United States
(12) Patent Application Publication
(10) Pub. No.: US 2026/0207564 A1
(43) Pub. Date: Jul. 23, 2026

(54) DOSAGE REGIME OF ORFORGLIPRON FOR TREATING A SUBJECT WITH TYPE 2 DIABETES (T2D), OBESITY, OR OVERWEIGHT WITH AT LEAST ONE WEIGHT RELATED COMORBIDITY

(71) Applicant: Eli Lilly and Company, Indianapolis, IN (US)

(72) Inventors: Mary Angelyn BETHEL, Carmel, IN (US); David Andrew COX, Batesville, IN (US); Stacy Jo KEDING, Brownsburg, IN (US); Manige KONIG, Carmel, IN (US); Rong LIU, Carmel, IN (US); Xiaosu MA, Mercer Island, WA (US); Edward John PRATT, Carmel, IN (US); Deborah Ann ROBINS, Fortville, IN (US)

(21) Appl. No.: 19/138,522

(22) PCT Filed: Dec. 12, 2023
(86) PCT No.: PCT/US2023/083545

§ 371 Date: Jun. 12, 2025

Related U.S. Application Data
(60) Provisional application No. 63/432,197, filed on Dec. 13, 2022.
(60) Provisional application No. 63/499,328, filed on May 1, 2023.

(51) Int. Cl. A61K 31/437; A61K 45/06; A61P 3/04; A61P 3/10

(52) U.S. Cl. CPC A61K 31/437; A61K 45/06; A61P 3/04; A61P 3/10

(57) ABSTRACT

Disclosed herein are methods for treating a subject with T2D, obesity, or overweight with at least one weight related comorbidity using a glucagon-like peptide-1 (GLP-1) receptor non-peptide agonist (NPA) by oral administration. Also disclosed herein are oral escalation doses for treating T2D, obesity, or overweight with at least one weight related comorbidity using the GLP-1 receptor NPA. Also disclosed are pharmaceutical unit dosages suitable for treating T2D, obesity, or overweight with at least one weight related comorbidity. Further disclosed herein are uses of the GLP-1 receptor NPA for the manufacture of a medicament for treating T2D, obesity, or overweight with at least one weight related comorbidity.

### CROSS REFERENCE TO RELATED APPLICATIONS

This application claims priority to U.S. Provisional Applications 63/432,197 filed Dec. 13, 2022 and 63/499,328 filed May 1, 2023, the contents of which are incorporated herein by reference in their entirety.

### TECHNICAL FIELD

Disclosed herein are methods for treating type 2 diabetes (T2D), obesity, or overweight with at least one weight related comorbidity using a glucagon-like peptide-1 (GLP-1) receptor non-peptide agonist (NPA) by oral administration. Also disclosed are oral dosing regimens for treating a subject with T2D, obesity, or overweight with at least one weight related comorbidity using the GLP-1 receptor NPA. Also disclosed are pharmaceutical unit dosages suitable for treating a subject with T2D, obesity, or overweight with at least one weight related comorbidity. Further disclosed are uses of the GLP-1 receptor NPA for the manufacture of a medicament for treating a subject with T2D, obesity, or overweight with at least one weight related comorbidity.

### BACKGROUND

Currently there are GLP-1 receptor agonists (RA) approved by health authorities for treating T2D, obesity, or overweight. Administration of peptides, such as GLP-1 RA, is often through parenteral routes rather than oral administration due to several barriers, such as enzymatic degradation in the gastrointestinal tract and intestinal mucosa, insufficient absorption from the intestinal mucosa, and first pass metabolism in the liver. An available oral GLP-1 RA product Rybelsus® contains a peptide as an active agent and its administration to patients comes with restrictions such as cumbersome food and water intake limitations. Patients prefer medications that they can integrate into their lives and daily routines and oral medications with no or less restrictive food and/or water intake limitations are generally preferred as they are simple to use. Thus, there is a need for improved methods for administering GLP-1 RA or GLP-1 receptor NPA.

### SUMMARY

Disclosed herein is a GLP-1 receptor NPA compound, Compound 1, having the following structure, or a pharmaceutically acceptable salt thereof, or a hydrate of the compound or the pharmaceutically acceptable salt:

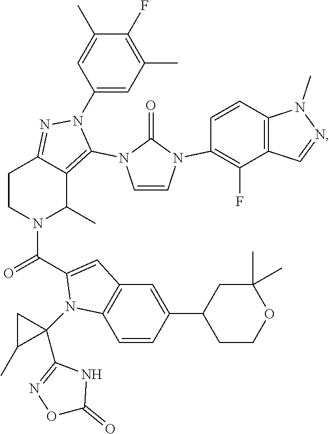 [SMILES: Cc1cc(-n2nc3c(c2-n2ccn(-c4ccc5c(cnn5C)c4F)c2=O)C(C)N(C(=O)c2cc4cc(C5CCOC(C)(C)C5)ccc4n2C2(c4noc(=O)[nH]4)CC2C)CC3)cc(C)c1F]

for treating a subject with T2D, obesity, or overweight with at least one weight related comorbidity. As used herein, treating a subject with obesity or overweight with at least one weight related comorbidity is also known as chronic weight management (CWM).

The specific doses and dose escalation schemes disclosed herein provide effective treatment options for T2D, obesity, or overweight with at least one weight related comorbidity while not being subject to the strict dosing limitations associated with other GLP-1 RA products.

### BRIEF DESCRIPTION OF DRAWINGS

FIG. 1 shows a clinical trial schema.

FIG. 2 shows a clinical trial schema.

FIG. 3 shows the effect of once daily OFG as compared to placebo on body weight. In FIG. 3, least square means are presented. Panel A shows the percentage change from baseline in body weight by week (efficacy estimand). Panel B shows the change from baseline in body weight (kg) by week. Panel C and D show the percentage of patients achieving ≥5%, ≥10%, and ≥15% weight reduction at 26 and 36 weeks, respectively. *=P-value <0.01 versus placebo. Doses 36 mg-1 and 36 mg-2 were combined for 36 mg and doses 45 mg-1 and 45 mg-2 were combined for 45 mg.

FIG. 4 shows a clinical trial schema.

FIG. 5 shows LS Mean change in HbA1c.

FIG. 6 shows LS Mean change in body weight.

FIG. 7 shows the percent of participants achieving HbA1c and weight loss targets.

### DESCRIPTION

GLP-1 RA has many actions in the body that may make them effective at lowering A1C (also known as “hemoglobin A1C” or “HbA1c”) or providing weight loss. However, available GLP1 RA products have various limitations such as the need for administration through injections or being subject to food and water intake limitations if administered orally. Compound 1 and the dosing schemes disclosed herein are effective in lowering the level of A1C and/or providing weight loss. Oral administration of Compound 1 provides advantages over injectable GLP-1 RA in that they are simple to use and can be integrated into patients' lives and daily routines. Dosing regimens described herein also provide advantages that Compound 1 can be taken at any time of the day with or without food, which provides improved convenience and reduces concerns about adequate drug absorption that affect orally delivered peptides.

Unfavorable side effects are often associated with a treatment of T2D, obesity, or overweight with at least one weight related comorbidity using a GLP-1 RA and achieving a desirable or more tolerable side effect profile has been a major challenge. The oral administration dosing schemes disclosed herein involve optimized dose escalations before reaching a maintenance dose resulting in reduced treatment related side effects.

In one embodiment, the dosing schemes disclosed herein are effective in treating a subject with T2D, obesity, or overweight with at least one weight related comorbidity, when used as an adjunct to diet and exercise, and have reduced treatment related side effects relative to when using a treatment dose without any escalation dose or with non-optimized escalation schedules.

In one embodiment, when used as an adjunct to diet and exercise, the dosing schemes disclosed herein are effective in treating a subject with T2D, obesity, or overweight with at least one weight related comorbidity, have reduced side effects relative to when using a treatment dose without any escalation dose or non-optimized escalation schedules, and are not subject to the strict food or water intake limitations associated with other oral GLP-1 RA products.

In one embodiment, when used as an adjunct to diet and exercise, the dosing schemes for oral administration of Compound 1 disclosed herein result in reduced treatment related adverse events such as nausea, vomiting, or constipation relative to when using an alternative dosing method such as using a treatment dose without one or more escalation doses or with non-optimized escalation schedules.

In one embodiment, when used as an adjunct to diet and exercise, a dosing regimen with a starting dose of between 0.7 mg and 1.2 mg of Compound 1 provides reduced treatment related side effects. In one embodiment, the starting dose is between 0.75 mg and 1 mg of Compound 1. In one embodiment, the starting dose is between 0.8 mg and 1 mg of Compound 1. In one embodiment, the starting dose is between 0.8 mg and 0.9 mg of Compound 1. In one embodiment, the starting dose is 1 mg of Compound 1. In one embodiment, each dose is a free acid equivalent amount of Compound 1.

In one embodiment, when used as an adjunct to diet and exercise, a dosing regimen with one or more escalation doses wherein each escalation dose is administered for 1, 2, 3, 4, 5, or 6 weeks provides reduced treatment related side effects. In one embodiment, each escalation dose is administered for 2, 3, 4, or 5 weeks. In one embodiment, each escalation dose is administered for 3, 4, or 5 weeks. In one embodiment, each escalation dose is administered for 4 weeks.

In one embodiment, when used as an adjunct to diet and exercise, a dosing regimen with a starting dose of between 0.7 mg and 1.2 mg of Compound 1, and one or more escalation doses wherein each escalation dose is administered for 1, 2, 3, 4, 5, or 6 weeks provides reduced treatment related side effects. In one embodiment, the dosing regimen comprises a starting dose of between 0.75 mg and 1 mg of Compound 1, and one or more escalation doses wherein each escalation dose is administered for 2, 3, 4, or 5 weeks. In one embodiment, the dosing regimen comprises a starting dose of between 0.8 mg and 1 mg of Compound 1, and one or more escalation doses wherein each escalation dose is administered for 4 weeks. In one embodiment, the dosing regimen comprises a starting dose of between 0.8 mg and 0.9 mg of Compound 1, and one or more escalation doses wherein each escalation dose is administered for 4 weeks. In one embodiment, the dosing regimen comprises a starting dose of 1 mg of Compound 1, and one or more escalation doses wherein each escalation dose is administered for 4 weeks. In one embodiment, each dose is a free acid equivalent amount of Compound 1.

In one embodiment, a method for treating a subject with type 2 diabetes (T2D), obesity, or overweight with at least one weight related comorbidity in need of such treatment, comprises: orally administering to the subject an effective amount of Compound 1, or a pharmaceutically acceptable salt thereof, or a hydrate of the compound or the pharmaceutically acceptable salt. In one embodiment, Compound 1, or a pharmaceutically acceptable salt thereof, or a hydrate of the compound or the pharmaceutically acceptable salt, is administered to the subject as an adjunct to diet and exercise.

In one embodiment of the method described above, the effective amount is selected from 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, and a combination of two or more of the listed doses. In one embodiment, each dose is a free acid equivalent amount of Compound 1. In one embodiment, each dose is administered once daily (QD). In one embodiment, one or more capsules comprising Compound 1, or a pharmaceutically acceptable salt thereof, or a hydrate of the compound or the pharmaceutically acceptable salt, is administered to the subject.

In one embodiment of the method described above, the effective amount is selected from 1 mg, 2 mg, 3 mg, 6 mg, 12 mg, 24 mg, 36 mg, and a combination of two or more of the listed doses. In one embodiment, each dose is a free acid equivalent amount of Compound 1. In one embodiment, each dose is administered QD. In one embodiment, one or more capsules comprising Compound 1, or a pharmaceutically acceptable salt thereof, or a hydrate of the compound or the pharmaceutically acceptable salt, is administered to the subject.

In one embodiment, one or more tablets each comprising an effective amount of Compound 1, or a pharmaceutically acceptable salt thereof, or a hydrate of the compound or the pharmaceutically acceptable salt, are administered to a subject, and wherein each effective amount is the amount equivalent to a corresponding capsule dose selected from 1 mg, 2 mg, 3 mg, 6 mg, 12 mg, 24 mg, 36 mg, and a combination of two or more of the listed doses. In one embodiment, each dose is a free acid equivalent amount of Compound 1. In one embodiment, each dose is administered once daily (QD). In one embodiment, the tablets are administered to the subject as an adjunct to diet and exercise.

In one embodiment of the method described above wherein one or more tablets are administered to the subject, the effective amount is selected from 0.7-1 mg, 2-3 mg, 4-6 mg, 8-12 mg, 9-20 mg, 13-30 mg, and a combination of two or more of the listed doses. In one embodiment, each dose is a free acid equivalent amount of Compound 1. In one embodiment, each dose is administered once daily (QD).

In one embodiment of the method described above wherein one or more tablets are administered to the subject, the effective amount is selected from 0.75-0.9 mg, 2.2-2.8 mg, 4.2-5.5 mg, 7.5-10 mg, 10-17 mg, 14-19 mg, and a combination of two or more of the listed doses. In one embodiment, each dose is a free acid equivalent amount of Compound 1. In one embodiment, each dose is administered once daily (QD).

In one embodiment of the method described above wherein one or more tablets are administered to the subject, the effective amount is selected from 0.8-0.9 mg, 2.3-2.7 mg, 4.5-5.3 mg, 8-9.5 mg, 11-16 mg, 14-18 mg, and a combination of two or more of the listed doses. In one embodiment, each dose is a free acid equivalent amount of Compound 1. In one embodiment, each dose is administered once daily (QD).

In one embodiment of the method described above, when used as an adjunct to diet and exercise, the method is for treating a subject with T2D mellitus for improved glycemic control; and wherein the oral administration results in a reduction of the A1C level of at least about 1.5% as compared to the A1C level of the subject at the start of the treatment.

In one embodiment of the method described above, when used as an adjunct to diet and exercise, the method is for treating a subject with obesity or overweight with at least one weight related comorbidity; and wherein the oral administration results in a weight loss of at least about 5% as compared to the weight of the subject at the start of the treatment.

In one embodiment of the method described above, Compound 1, or a pharmaceutically acceptable salt thereof, or a hydrate of the compound or the pharmaceutically acceptable salt, is administered in the form of a capsule, tablet, sachet, or granule; and wherein the capsule, tablet, sachet or granule comprises Compound 1, or a pharmaceutically acceptable salt thereof, or a hydrate of the compound or the pharmaceutically acceptable salt; and at least one pharmaceutically acceptable excipient.

In one embodiment, Compound 1, or a pharmaceutically acceptable salt thereof, or a hydrate of the compound or the pharmaceutically acceptable salt, is administered to a subject according to the following dosing regimen:
starting at a dose of 1 mg QD and administering at this dose for 4 weeks;
if additional glycemic or weight control is needed, then increasing to 3 mg QD and administering at this dose for 4 weeks if this dose is tolerated;
if additional glycemic or weight control is needed, then increasing to 6 mg QD and administering at this dose for 4 weeks if this dose is tolerated;
if additional glycemic or weight control is needed, then increasing to 12 mg QD and administering at this dose for 4 weeks if this dose is tolerated;
if additional glycemic or weight control is needed, then increasing to 24 mg QD and administering at this dose for 4 weeks if this dose is tolerated; and
if additional glycemic or weight control is needed, then increasing to 36 mg QD and administering at this dose as a maintenance dose for at least 4 weeks if this dose is tolerated;
wherein if a maximum tolerated dose (MTD) is reached at any of the 3 mg QD, 6 mg QD, 12 mg QD, 24 mg QD, and 36 mg QD, then dose escalation stops at the MTD dose; and
wherein the MTD dose is used as the maintenance dose and administered for at least 4 weeks.

In one embodiment, Compound 1, or a pharmaceutically acceptable salt thereof, or a hydrate of the compound or the pharmaceutically acceptable salt, is administered to a subject according to the following dosing regimen:
starting at a dose of 1 mg QD and administering at this dose for 4 weeks;
then increasing to 3 mg QD and administering at this dose for 4 weeks;
then increasing to 6 mg QD and administering at this dose for 4 weeks;
then increasing to 12 mg QD and administering at this dose for 4 weeks;
then increasing to 24 mg QD and administering at this dose for 4 weeks; and
then increasing to 36 mg QD and administering at this dose as a maintenance dose for at least 4 weeks. In one embodiment, each dose is a free acid equivalent amount of Compound 1.

In one embodiment, Compound 1, or a pharmaceutically acceptable salt thereof, or a hydrate of the compound or the pharmaceutically acceptable salt, is administered to a subject according to the following dosing regimen:
starting at a dose of 1 mg QD and administering at this dose for 4 weeks;
then increasing to 3 mg QD and administering at this dose for 4 weeks;
then increasing to 6 mg QD and administering at this dose for 4 weeks; and
then increasing to 12 mg QD and administering at this dose as a maintenance dose for at least 4 weeks. In one embodiment, each dose is a free acid equivalent amount of Compound 1.

In one embodiment, Compound 1, or a pharmaceutically acceptable salt thereof, or a hydrate of the compound or the pharmaceutically acceptable salt, is administered to a subject according to the following dosing regimen:
starting at a dose of 1 mg QD and administering at this dose for 4 weeks;
then increasing to 3 mg QD and administering at this dose for 4 weeks; and
then increasing to 6 mg QD and administering at this dose as a maintenance dose for at least 4 weeks. In one embodiment, each dose is a free acid equivalent amount of Compound 1.

In one embodiment, Compound 1, or a pharmaceutically acceptable salt thereof, or a hydrate of the compound or the pharmaceutically acceptable salt, is administered to a subject according to the following dosing regimen:
starting at a dose of 1 mg QD and administering at this dose for 4 weeks; and
then increasing to 3 mg QD and administering at this dose as a maintenance dose for at least 4 weeks. In one embodiment, each dose is a free acid equivalent amount of Compound 1.

In one embodiment of the dosing regimens described above, Compound 1, or a pharmaceutically acceptable salt thereof, or a hydrate of the compound or the pharmaceutically acceptable salt, is administered to the subject as an adjunct to diet and exercise.

In one embodiment of the method described above, the maintenance dose is administered QD for at least 16 weeks.

In one embodiment of the method described above, the maintenance dose is administered QD for at least 20 weeks.

In one embodiment of the method described above, the maintenance dose is administered QD for at least 32 weeks.

In one embodiment of the method described above, the maintenance dose is administered QD for at least 52 weeks.

In one embodiment of the method described above, the maintenance dose is administered QD for at least 84 weeks.

In one embodiment, Compound 1, or a pharmaceutically acceptable salt thereof, or a hydrate of the compound or the pharmaceutically acceptable salt, is administered to a subject in a tablet dosage form according to the following dosing regimen:
starting at a dose of 0.75-0.9 mg QD and administering at this dose for 4 weeks;
then increasing to 2.2-2.8 mg QD and administering at this dose for 4 weeks;
then increasing to 4.2-5.5 mg QD and administering at this dose for 4 weeks;
then increasing to 7.5-10 mg QD and administering at this dose for 4 weeks;
then increasing to 10.5-17 mg QD and administering at this dose for 4 weeks; and
then increasing to 13-19 mg QD and administering at this dose as a maintenance dose for at least 4 weeks.

In one embodiment, Compound 1, or a pharmaceutically acceptable salt thereof, or a hydrate of the compound or the pharmaceutically acceptable salt, is administered to a subject in a tablet dosage form according to the following dosing regimen:
starting at a dose of 0.75-0.9 mg QD and administering at this dose for 4 weeks;
then increasing to 2.2-2.8 mg QD and administering at this dose for 4 weeks;
then increasing to 4.2-5.5 mg QD and administering at this dose for 4 weeks; and
then increasing to 7.5-10 mg QD and administering at this dose as a maintenance dose for at least 4 weeks.

In one embodiment, Compound 1, or a pharmaceutically acceptable salt thereof, or a hydrate of the compound or the pharmaceutically acceptable salt, is administered to a subject in a tablet dosage form according to the following dosing regimen:
starting at a dose of 0.75-0.9 mg QD and administering at this dose for 4 weeks;
then increasing to 2.2-2.8 mg QD and administering at this dose for 4 weeks; and
then increasing to 4.2-5.5 mg QD and administering at this dose as a maintenance dose for 4 at least weeks.

In one embodiment, Compound 1, or a pharmaceutically acceptable salt thereof, or a hydrate of the compound or the pharmaceutically acceptable salt, is administered to a subject in a tablet dosage form according to the following dosing regimen:
starting at a dose of 0.75-0.9 mg QD and administering at this dose for 4 weeks; and
then increasing to 2.2-2.8 mg QD and administering at this dose as a maintenance dose for at least 4 weeks.

In one embodiment of the dosing regimens using a tablet dosage form described above, each dose is a free acid equivalent amount of Compound 1.

In one embodiment of the dosing regimens using a tablet dosage form described above, Compound 1, or a pharmaceutically acceptable salt thereof, or a hydrate of the compound or the pharmaceutically acceptable salt, is administered to the subject as an adjunct to diet and exercise.

In one embodiment of the method described above, there is no restriction on food or water intake by the subject within half an hour before or after the oral administration of Compound 1, or a pharmaceutically acceptable salt thereof, or a hydrate of the compound or the pharmaceutically acceptable salt to the subject.

In one embodiment of the method described above, the method is for treating a subject with T2D mellitus to improve glycemic control, as an adjunct to diet and exercise, wherein the oral administration results in a reduction of the A1C level of at least about 1.0% as compared to the starting A1C level of the subject at the start of the treatment (baseline). In one embodiment, the reduction of the A1C level is at least about 1.1%. In one embodiment, the reduction of the A1C level is at least about 1.2%. In one embodiment, the reduction of the A1C level is at least about 1.3%. In one embodiment, the reduction of the A1C level is at least about 1.4%. In one embodiment, the reduction of the A1C level is at least about 1.5%. In one embodiment, the reduction of the A1C level is at least about 1.6%. In one embodiment, the reduction of the A1C level is at least about 1.7%. In one embodiment, the reduction of the A1C level is at least about 1.8%. In one embodiment, the reduction of the A1C level is at least about 1.9%. In one embodiment, the reduction of the A1C level is at least about 2.0%. In one embodiment, the reduction of the A1C level is at least about 2.1%. In one embodiment, the reduction of the A1C level is at least about 2.2%.

In one embodiment of the method described above, the method is for treating a subject with obesity or overweight with at least one weight related comorbidity, as an adjunct to diet and exercise, wherein the oral administration results in a weight loss of at least about 5% as compared to the weight of the subject at the start of the treatment (baseline). In one embodiment, the weight loss is at least about 6%. In one embodiment, the weight loss is at least about 7%. In one embodiment, the weight loss is at least about 7.5%. In one embodiment, the weight loss is at least about 8%. In one embodiment, the weight loss is at least about 9%. In one embodiment, the weight loss is at least about 10%. In one embodiment, the weight loss is at least about 11%. In one embodiment, the weight loss is at least about 12%.

In one embodiment of the method described above, the method is for treating a subject with obesity or overweight with at least one weight related comorbidity, as an adjunct to diet and exercise, wherein the oral administration results in a weight loss of at least about 3.5 kg as compared to the weight of the subject at the start of the treatment (baseline). In one embodiment, the weight loss is at least about 4 kg. In one embodiment, the weight loss is at least about 4.5 kg. In one embodiment, the weight loss is at least about 5 kg. In one embodiment, the weight loss is at least about 5.5 kg. In one embodiment, the weight loss is at least about 6 kg. In one embodiment, the weight loss is at least about 6.5 kg. In one embodiment, the weight loss is at least about 7 kg. In one embodiment, the weight loss is at least about 7.5 kg. In one embodiment, the weight loss is at least about 8 kg. In one embodiment, the weight loss is at least about 8.5 kg. In one embodiment, the weight loss is at least about 9 kg. In one embodiment, the weight loss is at least about 9.5 kg. In one embodiment, the weight loss is at least about 10 kg.

In one embodiment of the method described above, when Compound 1, or a pharmaceutically acceptable salt thereof, or a hydrate of the compound or the pharmaceutically acceptable salt, is administered to an adult subject with T2D, obesity, or overweight with at least one weight related comorbidity at increased cardiovascular risk, the oral administration of Compound 1, or a pharmaceutically acceptable salt thereof, or a hydrate of the compound or the pharmaceutically acceptable salt, is non-inferior as compared to an insulin glargine treatment (with a non-inferiority margin of 1.8) in the occurrence of MACE-4 events. In one embodiment, Compound 1, or a pharmaceutically acceptable salt thereof, or a hydrate of the compound or the pharmaceutically acceptable salt, is administered to the subject as an adjunct to diet and exercise.

In one embodiment of the method described above, when Compound 1, or a pharmaceutically acceptable salt thereof, or a hydrate of the compound or the pharmaceutically acceptable salt, is administered to an adult subject with T2D, obesity, or overweight with at least one weight related comorbidity at increased cardiovascular risk, the oral administration of Compound 1, or a pharmaceutically acceptable salt thereof, or a hydrate of the compound or the pharmaceutically acceptable salt, is superior to an insulin glargine treatment (with a non-inferiority margin of 1.8) in the occurrence of MACE-4 events.

In one embodiment of the method described above, a method for treating a subject with type 2 diabetes mellitus in need of such treatment, comprises:
orally administering Compound 1, or a pharmaceutically acceptable salt thereof, or a hydrate of the compound or the pharmaceutically acceptable salt, to the subject at a maintenance dose selected from 3 mg, 6 mg, 12 mg, and 36 mg;
wherein, before a maintenance dose is administered, optional escalation doses per the following regimen are administered:
starting at a dose of 1-2 mg QD for days or weeks;
optionally, if additional glycemic control is needed, increasing the dose to 2-5 mg QD and administering at this dose for days or weeks if this dose is tolerated;
optionally, if additional glycemic control is needed, increasing the dose to 5-10 mg QD and administering at this dose for days or weeks if this dose is tolerated;
optionally, if additional glycemic control is needed, increasing the dose to 10-20 mg QD and administering at this dose for days or weeks if this dose is tolerated;
optionally, if additional glycemic control is needed, increasing the dose to 20-30 mg QD and administering at this dose for days or weeks if this dose is tolerated; and
optionally, if additional glycemic control is needed, increasing the dose to 30-40 mg QD and administering at this dose for days or weeks if this dose is tolerated;
wherein if a maximum tolerated dose (MTD) is reached at any of the 2-5 mg QD, 5-10 mg QD, 10-20 mg QD, 20-30 mg QD, and 30-40 mg QD dose, then dose escalation stops at the MTD dose; and
wherein the MTD dose is used as the maintenance dose.

In one embodiment of the method described above for treating a subject with T2D to improve glycemic control, the maintenance dose is 3 mg QD; and wherein, before the maintenance dose is administered,
a starting dose at 1 mg QD is administered for 2, 3, 4, 5, or 6 weeks. In one embodiment, the starting dose of 1 mg QD is administered for 4 weeks.

In one embodiment of the method described above for treating a subject with T2D to improve glycemic control, the maintenance dose is 12 mg QD; and wherein, before the maintenance dose is administered,
a starting dose at 1 mg QD is administered for 2, 3, 4, 5, or 6 weeks;
then increasing the dose to 3 mg QD and administering at this dose for 2, 3, 4, 5, or 6 weeks; and
then increasing the dose to 6 mg QD and administering at this dose for 2, 3, 4, 5, or 6 weeks.

In one embodiment of the method described above for treating a subject with T2D to improve glycemic control, the maintenance dose is 36 mg QD; and wherein, before the maintenance dose is administered,
a starting dose at 1 mg QD is administered for 2, 3, 4, 5, or 6 weeks;
then increasing the dose to 3 mg QD and administering at this dose for 2, 3, 4, 5, or 6 weeks;
then increasing the dose to 6 mg QD and administering at this dose for 2, 3, 4, 5, or 6 weeks;
then increasing the dose to 12 mg QD and administering at this dose for 2, 3, 4, 5, or 6 weeks; and
then increasing the dose to 24 mg QD and administering at this dose for 2, 3, 4, 5, or 6 weeks.

In one embodiment of the method described above for treating a subject with T2D to improve glycemic control, Compound 1, or a pharmaceutically acceptable salt thereof, or a hydrate of the compound or the pharmaceutically acceptable salt, is administered to the subject at the maintenance dose QD or the MTD dose QD for days, weeks or years.

In one embodiment of the method described above for treating a subject with T2D to improve glycemic control, Compound 1, or a pharmaceutically acceptable salt thereof, or a hydrate of the compound or the pharmaceutically acceptable salt, is administered to the subject at the maintenance dose QD or the MTD dose QD for at least 20, 28, 32, 36, 40, 44, 48, 50, 52, 60, 64, 68, 84, 92, 96, or 100 weeks.

In one embodiment of the method described above for treating a subject with T2D to improve glycemic control, the oral administration results in a reduction of the A1C level of at least about 1.5% as compared to the A1C level of the subject at the start of the treatment. In one embodiment, the reduction of the A1C level is at least about 1.6%. In one embodiment, the reduction of the A1C level is at least about 1.7%. In one embodiment, the reduction of the A1C level is at least about 1.8%. In one embodiment, the reduction of the A1C level is at least about 1.9%. In one embodiment, the reduction of the A1C level is at least about 2.0%.

In one embodiment, a method for treating a subject with obesity or overweight with at least one weight related comorbidity comprises:
orally administering Compound 1, or a pharmaceutically acceptable salt thereof, or a hydrate of the compound or the pharmaceutically acceptable salt, to the subject at a maintenance dose selected from 6 mg, 12 mg, and 36 mg;
wherein, before a maintenance dose is administered, optional escalation doses per the following regimen are administered:
starting at a dose of 1-2 mg QD for days or weeks;
optionally, if additional weight control is needed, increasing the dose to 2-5 mg QD and administering at this dose for days or weeks if this dose is tolerated;
optionally, if additional weight control is needed, increasing the dose to 5-10 mg QD and administering at this dose for days or weeks if this dose is tolerated;
optionally, if additional weight control is needed, increasing the dose to 10-20 mg QD and administering at this dose for days or weeks if this dose is tolerated; and
optionally, if additional weight control is needed, increasing the dose to 20-30 mg QD and administering at this dose for days or weeks if this dose is tolerated;
wherein if a maximum tolerated dose (MTD) is reached at any of the optional 2-5 mg QD, 5-10 mg QD, 10-20 mg QD, and 20-30 mg QD, then dose escalation stops at the MTD dose; and
wherein the MTD dose is used as a maintenance dose.

In one embodiment of the method described above for treating a subject with obesity or overweight with at least one weight related comorbidity, the maintenance dose is 6 mg QD; and wherein, before the maintenance dose is administered,
a starting dose at 1 mg QD is administered for 2, 3, 4, 5, or 6 weeks; and
then increasing the dose to 3 mg QD and administering at this dose for 2, 3, 4, 5, or 6 weeks.

In one embodiment of the method described above for treating a subject with obesity or overweight with at least one weight related comorbidity, the maintenance dose is 12 mg QD; and wherein, before the maintenance dose is administered,
a starting dose at 1 mg QD is administered for 2, 3, 4, 5, or 6 weeks;
then increasing the dose to 3 mg QD and administering at this dose for 2, 3, 4, 5, or 6 weeks; and
then increasing the dose to 6 mg QD and administering at this dose for 2, 3, 4, 5, or 6 weeks.

In one embodiment of the method described above for treating a subject with obesity or overweight with at least one weight related comorbidity, the maintenance dose is 36 mg QD; and wherein, before the maintenance dose is administered,
a starting dose at 1 mg QD is administered for 2, 3, 4, 5, or 6 weeks;
then increasing the dose to 3 mg QD and administering at this dose for 2, 3, 4, 5, or 6 weeks;
then increasing the dose to 6 mg QD and administering at this dose for 2, 3, 4, 5, or 6 weeks;
then increasing the dose to 12 mg QD and administering at this dose for 2, 3, 4, 5, or 6 weeks; and
then increasing the dose to 24 mg QD and administering at this dose for 2, 3, 4, 5, or 6 weeks.

In one embodiment of the method described herein for treating a subject with obesity or overweight with at least one weight related comorbidity, Compound 1, or a pharmaceutically acceptable salt thereof, or a hydrate of the compound or the pharmaceutically acceptable salt, is administered to the subject at the maintenance dose QD or the MTD dose QD for at least 20, 28, 32, 36, 40, 44, 48, 50, 52, 60, 64, 68, 84, 92, 96, or 100 weeks.

In one embodiment of the dosing regimens described above for treating obesity or overweight with at least one weight related comorbidity, Compound 1, or a pharmaceutically acceptable salt thereof, or a hydrate of the compound or the pharmaceutically acceptable salt, is administered to the subject as an adjunct to diet and exercise.

In one embodiment of the method described above for treating a subject with obesity or overweight with at least one weight related comorbidity, the oral administration results in a weight loss of at least about 5% as compared to the weight of the subject at the start of the treatment. In one embodiment, the weight loss is at least about 6%. In one embodiment, the weight loss is at least about 7%. In one embodiment, the weight loss is at least about 7.5%. In one embodiment, the weight loss is at least about 8%. In one embodiment, the weight loss is at least about 9%. In one embodiment, the weight loss is at least about 10%.

In one embodiment of the method described herein, Compound 1 has the following structure (Compound 1a):

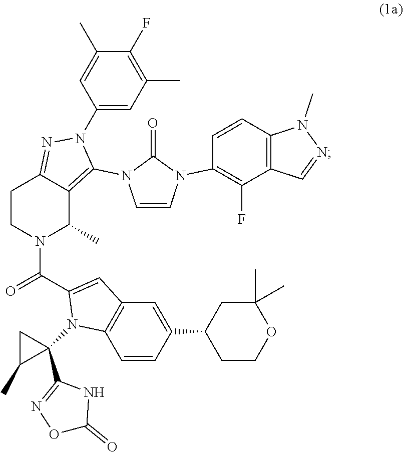 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

or a pharmaceutically acceptable salt thereof, or a hydrate of the compound or the pharmaceutically acceptable salt.

In one embodiment of the method described herein, a hemicalcium salt of Compound 1a is used as the active agent.

In one embodiment of the method described herein, a hemicalcium salt hydrate of Compound 1 having the following structure (Compound 1b) is used as the active agent:

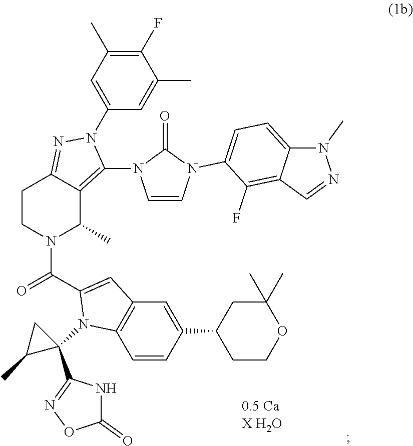

wherein X is a whole or a decimal number between 0.1 and 2. In one embodiment, X is 0.5 (hemihydrate). In one embodiment, X is 1 (monohydrate). In one embodiment, X is 2 (dihydrate).

Also disclosed herein is a treatment of a subject with T2D, obesity, or overweight with at least one weight related comorbidity using Compound 1, or a pharmaceutically acceptable salt thereof, or a hydrate of the compound or the pharmaceutically acceptable salt, in combination with a second active agent.

In one embodiment, a method for treating a subject with T2D, obesity, or overweight with at least one weight related comorbidity comprises administering Compound 1, or a pharmaceutically acceptable salt thereof, or a hydrate of the compound or the pharmaceutically acceptable salt, using a dosing regimen disclosed herein, in combination with a second active agent. In one embodiment, the second active agent is selected from the group consisting of an amylin receptor non-peptide agonist, a glucagon receptor non-peptide agonist, a glucose-dependent insulinotropic polypeptide (GIP) non-peptide agonist, a peptide tyrosine-tyrosine (PYY) non-peptide agonist, and a mixture thereof. In one embodiment, the second active agent is co-administered with Compound 1, or a pharmaceutically acceptable salt thereof, or a hydrate of the compound or the pharmaceutically acceptable salt, concomitantly. In one embodiment, the second active agent is co-administered with Compound 1, or a pharmaceutically acceptable salt thereof, or a hydrate of the compound or the pharmaceutically acceptable salt, sequentially.

In one embodiment, the combination administration of Compound 1, or a pharmaceutically acceptable salt thereof, or a hydrate of the compound or the pharmaceutically acceptable salt, and the second active agent results in a weight loss of at least about 10%, or more specifically, a weight loss of at least about 15%, or even more specifically, a weight loss of at least about 20%; as compared to the weight of the subject at the start of the treatment.

In one embodiment, the combination administration of Compound 1, or a pharmaceutically acceptable salt thereof, or a hydrate of the compound or the pharmaceutically acceptable salt, and the second active agent results in a reduction of the A1C level of at least about 2.0%; or more specifically, a reduction of the A1C level of at least about 2.1%; or more specifically, a reduction of the A1C level of at least about 2.2%; or even more specifically, a reduction of the A1C level of at least about 2.5%; as compared to the A1C level of the subject at the start of the treatment.

In one embodiment, a method for treating a subject with type 2 diabetes (T2D), obesity, or overweight with at least one weight related comorbidity comprises:
orally administering to the subject a free acid or a hemicalcium salt of Compound 1a having the following structure:

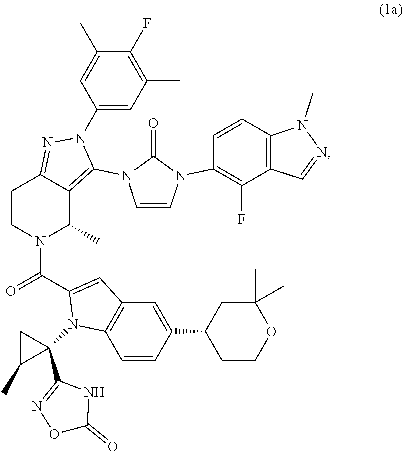 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

according to the following dosing regimen:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks. In one embodiment, the maintenance dose is administered for at least 20 weeks. In one embodiment, the maintenance dose is administered for at least 32 weeks. In one embodiment, the maintenance dose is administered for at least 52 weeks. In one embodiment, the maintenance dose is administered for at least 84 weeks.

In one embodiment, a method for treating a subject with type 2 diabetes (T2D), obesity, or overweight with at least one weight related comorbidity comprises:
orally administering to the subject a free acid or a hemicalcium salt of Compound 1a according to the following dosing regimen:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks. In one embodiment, the maintenance dose is administered for at least 28 weeks. In one embodiment, the maintenance dose is administered for at least 40 weeks. In one embodiment, the maintenance dose is administered for at least 60 weeks. In one embodiment, the maintenance dose is administered for at least 92 weeks.

In one embodiment, a method for treating a subject with type 2 diabetes (T2D), obesity, or overweight with at least one weight related comorbidity comprises:
orally administering to the subject a free acid or a hemicalcium salt of Compound 1a according to the following dosing regimen:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks. In one embodiment, the maintenance dose is administered for at least 32 weeks. In one embodiment, the maintenance dose is administered for at least 44 weeks. In one embodiment, the maintenance dose is administered for at least 64 weeks. In one embodiment, the maintenance dose is administered for at least 96 weeks.

In one embodiment, a method for treating a subject with type 2 diabetes (T2D), obesity, or overweight with at least one weight related comorbidity comprises:
orally administering to the subject a free acid or a hemicalcium salt of Compound 1a according to the following dosing regimen:
starting at a dose of 1 mg QD, and administering at this dose for 4 weeks; and
then increasing the dose to 3 mg QD, and administering at this dose as a maintenance dose for at least 4 weeks. In one embodiment, the maintenance dose is administered for at least 36 weeks. In one embodiment, the maintenance dose is administered for at least 48 weeks. In one embodiment, the maintenance dose is administered for at least 68 weeks. In one embodiment, the maintenance dose is administered for at least 100 weeks.

In one embodiment of the method described above, each dose is a free acid equivalent amount of Compound 1a.

In one embodiment of the method as described above, the free acid or hemicalcium salt of Compound 1a is administered to the subject as an adjunct to diet and exercise.

In one embodiment, the above method having a maintenance dose of 3 mg QD, 6 mg QD, 12 mg QD, or 36 mg QD is for improved glycemic control in a subject with T2D mellitus; wherein the oral administration of a free acid or a hemicalcium salt of Compound 1a results in a reduction of the subject's HbA1c level of at least 1.0%. In one embodiment, the reduction of the HbA1c level is at least 1.1%. In one embodiment, the reduction of the HbA1c level is at least 1.2%. In one embodiment, the reduction of the HbA1c level is at least 1.3%. In one embodiment, the reduction of the HbA1c level is at least 1.4%. In one embodiment, the reduction of the HbA1c level is at least 1.5%.

In one embodiment of the above method for improved glycemic control in a subject with T2D mellitus, the oral administration of a free acid or a hemicalcium salt of Compound 1a additionally results in a weight reduction of at least 5%. In one embodiment, the weight reduction is at least 6%. In one embodiment, the weight reduction is at least 7%. In one embodiment, the weight reduction is at least 8%. In one embodiment, the weight reduction is at least 9%. In one embodiment, the weight reduction is at least 10%.

In one embodiment, the above method having a maintenance dose of 3 mg QD, 6 mg QD, 12 mg QD, or 36 mg QD is for treating a subject with obesity or overweight with at least one weight related comorbidity; wherein the oral administration of a free acid or a hemicalcium salt of Compound 1a results in a weight reduction of at least 5%. In one embodiment, the weight reduction is at least 6%. In one embodiment, the weight reduction is at least 7%. In one embodiment, the weight reduction is at least 8%. In one embodiment, the weight reduction is at least 9%. In one embodiment, the weight reduction is at least 10%.

In one embodiment, the above method having a maintenance dose of 3 mg QD, 6 mg QD, 12 mg QD, or 36 mg QD is for treating a subject with obesity or overweight with at least one weight related comorbidity; wherein the oral administration of a free acid or a hemicalcium salt of Compound 1a results in a weight reduction of at least 5 kg. In one embodiment, the weight reduction is at least 6 kg. In one embodiment, the weight reduction is at least 7 kg. In one embodiment, the weight reduction is at least 8 kg. In one embodiment, the weight reduction is at least 9 kg. In one embodiment, the weight reduction is at least 10 kg.

In one embodiment of the method as described above, the free acid or the hemicalcium salt of Compound 1a is administered to the subject as an adjunct to diet and exercise.

In one embodiment of the above method having a maintenance dose of 3 mg QD, 6 mg QD, 12 mg QD, or 36 mg QD, the subject is not restricted from food or water intake within 30 minutes before or after each oral administration of the free acid or hemicalcium salt of Compound 1a.

In one embodiment, the subject is not restricted from food or water intake within 45 minutes before or after each oral administration of the free acid or hemicalcium salt of Compound 1a.

In one embodiment, the subject is not restricted from food or water intake within 1 hour before or after each oral administration of the free acid or hemicalcium salt of Compound 1a.

In one embodiment of the above method having a maintenance dose of 3 mg QD, 6 mg QD, 12 mg QD, or 36 mg QD, oral administration of one or more escalation doses prior to administration of a maintenance dose results in less treatment related side effects.

In one embodiment, oral administration of the free acid or hemicalcium salt of Compound 1a is co-administered with a second active agent concomitantly or sequentially.

In one embodiment, the second active agent is selected from the group consisting of: an amylin receptor non-peptide agonist, a glucagon receptor non-peptide agonist, a glucose-dependent insulinotropic polypeptide (GIP) non-peptide agonist, a peptide tyrosine-tyrosine (PYY) non-peptide agonist, and a mixture thereof.

In one embodiment, disclosed herein is a use of Compound 1, or a pharmaceutically acceptable salt thereof, or a hydrate of the compound or the pharmaceutically acceptable salt, for the treatment of a subject with type 2 diabetes (T2D), obesity, overweight with at least one weight related comorbidity in need of such treatment, wherein:
Compound 1, or a pharmaceutically acceptable salt thereof, or a hydrate of the compound or the pharmaceutically acceptable salt, is for oral administration to the subject;
at a dose selected from 1 mg, 3 mg, 6 mg, 12 mg, 24 mg, 36 mg, and a combination of two or more of the listed doses.

In one embodiment of the use, each administered dose is a QD dose.

In one embodiment of the use, Compound 1, or a pharmaceutically acceptable salt thereof, or a hydrate of the compound or the pharmaceutically acceptable salt, is for oral administration to a subject according to the following schedule:
starting at a dose of 1-2 mg QD and administering at this dose for 2, 3, 4, 5, or 6 weeks;
if additional glycemic or weight control is needed, then increasing the dose to 2-5 mg QD and administering at this dose for 2, 3, 4, 5, or 6 weeks if this dose is tolerated;
if additional glycemic or weight control is needed, then increasing the dose to 5-10 mg QD and administering at this dose for 2, 3, 4, 5, or 6 weeks if this dose is tolerated;
if additional glycemic or weight control is needed, then increasing the dose to 10-20 mg QD and administering at this dose for 2, 3, 4, 5, or 6 weeks if this dose is tolerated;
if additional glycemic or weight control is needed, then increasing the dose to 20-30 mg QD and administering at this dose for 2, 3, 4, 5, or 6 weeks if this dose is tolerated; and
if additional glycemic or weight control is needed, then increasing the dose to 30-40 mg QD and administering at this dose as a maintenance dose for at least 4 weeks if this dose is tolerated;
wherein if a maximum tolerated dose (MTD) is reached at any of the 2-5 mg QD, 5-10 mg QD, 10-20 mg QD, and 20-30 mg QD, then dose escalation stops at the MTD dose without further escalation to the next dose level; and
wherein the MTD dose is used as a maintenance dose and administered for at least 4 weeks.

In one embodiment of the use, Compound 1, or a pharmaceutically acceptable salt thereof, or a hydrate of the compound or the pharmaceutically acceptable salt, is for oral administration to a subject according to the following schedule:
starting at a dose of 1 mg QD and administering at this dose for 4 weeks;
if additional glycemic or weight control is needed, then increasing the dose to 3 mg QD and administering at this dose for 4 weeks if this dose is tolerated;
if additional glycemic or weight control is needed, then increasing the dose to 6 mg QD and administering at this dose for 4 weeks if this dose is tolerated;
if additional glycemic or weight control is needed, then increasing the dose to 12 mg QD and administering at this dose for 4 weeks if this dose is tolerated;
if additional glycemic or weight control is needed, then increasing the dose to 24 mg QD and administering at this dose for 4 weeks if this dose is tolerated; and
if additional glycemic or weight control is needed, then increasing the dose to 36 mg QD and administering at this dose as a maintenance dose for at least 4 weeks if this dose is tolerated;
wherein if a maximum tolerated dose (MTD) is reached at any of the 3 mg QD, 6 mg QD, 12 mg QD, 24 mg QD, and 36 mg QD, then dose escalation stops at the MTD dose without further escalation to the next dose level; and
wherein the MTD dose is used as the maintenance dose and administered for at least 4 weeks.

In one embodiment, disclosed herein is a use of Compound 1, or a pharmaceutically acceptable salt thereof, or a hydrate of the compound or the pharmaceutically acceptable salt, for the treatment of a subject with T2D, obesity, or overweight with at least one weight related comorbidity according to the following oral administration schedule:
starting at a dose of 1 mg QD, for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks.

In one embodiment, the maintenance dose of 36 mg QD is administered for at least 20 weeks. In one embodiment, the maintenance dose of 36 mg QD is administered for at least 32 weeks. In one embodiment, the maintenance dose of 36 mg QD is administered for at least 52 weeks.

In one embodiment, disclosed herein is a use of Compound 1, or a pharmaceutically acceptable salt thereof, or a hydrate of the compound or the pharmaceutically acceptable salt, for the treatment of a subject with T2D, obesity, or overweight with at least one weight related comorbidity according to the following oral administration schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks.

In one embodiment, the maintenance dose of 12 mg QD is administered for at least 28 weeks. In one embodiment, the maintenance dose of 12 mg QD is administered for at least 40 weeks. In one embodiment, the maintenance dose of 12 mg QD is administered for at least 60 weeks.

In one embodiment, disclosed herein is a use of Compound 1, or a pharmaceutically acceptable salt thereof, or a hydrate of the compound or the pharmaceutically acceptable salt, for the treatment of a subject with T2D, obesity, or overweight with at least one weight related comorbidity according to the following oral administration schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks.

In one embodiment, the maintenance dose of 6 mg QD is administered for at least 32 weeks. In one embodiment, the maintenance dose of 6 mg QD is administered for at least 44 weeks. In one embodiment, the maintenance dose of 6 mg QD is administered for at least 64 weeks.

In one embodiment, disclosed herein is a use of Compound 1, or a pharmaceutically acceptable salt thereof, or a hydrate of the compound or the pharmaceutically acceptable salt, for the treatment of a subject with T2D, obesity, or overweight with at least one weight related comorbidity according to the following oral administration schedule:
starting at a dose of 1 mg QD, and administering at this dose for 4 weeks; and
then increasing the dose to 3 mg QD, and administering at this dose as a maintenance dose for at least 4 weeks.

In one embodiment, the maintenance dose of 3 mg QD is administered for at least 36 weeks. In one embodiment, the maintenance dose of 3 mg QD is administered for at least 48 weeks. In one embodiment, the maintenance dose of 3 mg QD is administered for at least 68 weeks.

In one embodiment of the uses described above, each dose is a free acid equivalent amount of Compound 1.

In one embodiment of the uses described above, Compound 1, or a pharmaceutically acceptable salt thereof, or a hydrate of the compound or the pharmaceutically acceptable salt, is administered to the subject as an adjunct to diet and exercise.

In one embodiment, the use is for treating a subject with T2D mellitus to improve glycemic control, wherein the administration results in a reduction of the A1C level of at least about 1.5% as compared to the A1C level of the subject at the start of the treatment. In one embodiment, the reduction of the A1C level is at least about 1.6%. In one embodiment, the reduction of the A1C level is at least about 1.7%. In one embodiment, the reduction of the A1C level is at least about 1.8%. In one embodiment, the reduction of the A1C level is at least about 1.9%. In one embodiment, the reduction of the A1C level is at least about 2.0%.

In one embodiment, the use is for treating a subject with obesity or overweight with at least one weight related comorbidity, wherein the oral administration results in a weight loss of at least about 5% as compared to the weight of the subject at the start of the treatment. In one embodiment, the weight loss is at least about 6%. In one embodiment, the weight loss is at least about 7%. In one embodiment, the weight loss is at least about 7.5%. In one embodiment, the weight loss is at least about 8%. In one embodiment, the weight loss is at least about 9%. In one embodiment, the weight loss is at least about 10%.

In one embodiment of the use as described above, oral administration of Compound 1, or a pharmaceutically acceptable salt thereof, or a hydrate of the compound or the pharmaceutically acceptable salt, is an adjunct to diet and exercise.

In one embodiment, disclosed herein is a use of Compound 1, or a pharmaceutically acceptable salt thereof, or a hydrate of the compound or the pharmaceutically acceptable salt, for the manufacture of a medicament for treating a subject with T2D, obesity, or overweight with at least one weight related comorbidity;
wherein the medicament is for oral administration of Compound 1, or a pharmaceutically acceptable salt thereof, or a hydrate of the compound or the pharmaceutically acceptable salt to the subject
at a once daily dose selected from 1 mg, 2 mg, 3 mg, 6 mg, 12 mg, 24 mg, 36 mg, and a combination thereof.

In one embodiment of the use described above, the medicament is for oral administration of Compound 1, or a pharmaceutically acceptable salt thereof, or a hydrate of the compound or the pharmaceutically acceptable salt to the subject according to the following dosing schedule:
starting at a dose of 1 mg QD, and administering at this dose for 4 weeks; and
then increasing the dose to 3 mg QD, and administering at this dose as a maintenance dose for at least 4 weeks.

In one embodiment of the uses described above, the medicament is for oral administration of Compound 1, or a pharmaceutically acceptable salt thereof, or a hydrate of the compound or the pharmaceutically acceptable salt to the subject according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks.

In one embodiment of the uses described above, the medicament is for oral administration of Compound 1, or a pharmaceutically acceptable salt thereof, or a hydrate of the compound or the pharmaceutically acceptable salt to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks.

In one embodiment of the uses described above, the medicament is for oral administration of Compound 1, or a pharmaceutically acceptable salt thereof, or a hydrate of the compound or the pharmaceutically acceptable salt to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks.

In one embodiment, disclosed herein is pharmaceutical unit dosage comprising from 1 mg to 36 mg of Compound 1, or a pharmaceutically acceptable salt thereof, or a hydrate of the compound or the pharmaceutically acceptable salt;
wherein the unit dosage is suitable for oral administration for treating a subject with T2D, obesity, or overweight with at least one weight related comorbidity; and
wherein multiple unit dosages each comprising a dose selected from 1 mg, 2 mg, 3 mg, 6 mg, 12 mg, 24 mg, 36 mg, and a combination thereof, are administered to the subject.

In one embodiment, multiple unit dosages are administered to a subject according to the following dosing schedule:
starting at a unit dosage of 1 mg QD, and administering at this dosage for 4 weeks; and
then increasing the unit dosage to 3 mg QD, and administering at this dosage as a maintenance dosage for at least 4 weeks.

In one embodiment of the unit dosage described above, multiple unit dosages are administered to the subject according to the following dosing schedule:
starting at a unit dosage of 1 mg QD, and administering at this dosage for 4 weeks;
then increasing the unit dosage to 3 mg QD, and administering at this dosage for 4 weeks; and
then increasing the unit dosage to 6 mg QD, and administering at this dosage as a maintenance dosage for at least 4 weeks.

In one embodiment of the unit dosage described above, multiple unit dosages are administered to the subject according to the following dosing schedule:
starting at a unit dosage of 1 mg QD, and administering at this dosage for 4 weeks;
then increasing the unit dosage to 3 mg QD, and administering at this dosage for 4 weeks;
then increasing the unit dosage to 6 mg QD, and administering at this dosage for 4 weeks; and
then increasing the unit dosage to 12 mg QD, as a maintenance unit dosage, and administering at this dosage as a maintenance dosage for at least 4 weeks.

In one embodiment of the unit dosage described above, multiple unit dosages are administered to the subject according to the following dosing schedule:
starting at a unit dosage of 1 mg QD, and administering at this dosage for 4 weeks;
then increasing the unit dosage to 3 mg QD, and administering at this dosage for 4 weeks;
then increasing the unit dosage to 6 mg QD, and administering at this dosage for 4 weeks;
then increasing the unit dosage to 12 mg QD, and administering at this dosage for 4 weeks;
then increasing the unit dosage to 24 mg QD, and administering at this dosage for 4 weeks; and
then increasing the unit dosage to 36 mg QD, and administering at this dosage as a maintenance dosage for at least 4 weeks.

In one embodiment of the use or unit dosage described above, each dose or unit dosage is a free acid equivalent amount of Compound 1.

In one embodiment of the use or unit dosage described above, the doses or unit dosages are administered to the subject for a total treatment period selected from at least 40 weeks, at least 52 weeks, at least 72 weeks, and at least 104 weeks; wherein the total treatment period comprises dose escalation period and dose maintenance period.

In one embodiment of the use or unit dosage described above, the doses or unit dosages are administered to the subject as an adjunct to diet and exercise.

In one embodiment of the use or unit dosage described above, Compound 1, or a pharmaceutically acceptable salt thereof, or a hydrate of the compound or the pharmaceutically acceptable salt, is a hemicalcium salt of Compound 1a or a hydrate thereof.

In one embodiment, disclosed herein is a use of a free acid or a hemicalcium salt of Compound 1a for the manufacture of a medicament for treating a subject with T2D, obesity, or overweight with at least one weight related comorbidity;
wherein the medicament is for oral administration of the free acid or the hemicalcium salt of Compound 1a to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks.

In one embodiment, a free acid or a hemicalcium salt of Compound 1a is used for the manufacture of one or more unit dosages for treating a subject with T2D, obesity, or overweight with at least one weight related comorbidity;
wherein each unit dosage comprises 1 mg, 3 mg, 6 mg, or 12 mg of the free acid or the hemicalcium salt of Compound 1a; and
wherein multiple unit dosages are administered to the subject according to the following dosing schedule:
starting at a unit dosage of 1 mg QD, and administering at this dosage for 4 weeks;
then increasing the unit dosage to 3 mg QD, and administering at this dosage for 4 weeks;
then increasing the unit dosage to 6 mg QD, and administering at this dosage for 4 weeks; and
then increasing the unit dosage to 12 mg QD, and administering at this dosage as a maintenance dosage for at least 4 weeks.

In one embodiment is a use of a free acid or a hemicalcium salt of Compound 1a for the manufacture of a medicament for treating a subject with T2D, obesity, or overweight with at least one weight related comorbidity;
wherein the medicament is for oral administration of the free acid or the hemicalcium salt of Compound 1a to the subject according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks.

In one embodiment is a use of a free acid or a hemicalcium salt of Compound 1a for the manufacture of a medicament for treating a subject with T2D, obesity, or overweight with at least one weight related comorbidity;
wherein the medicament is for oral administration of the free acid or the hemicalcium salt of Compound 1a to the subject according to the following dosing schedule:
starting at a dose of 1 mg QD, and administering at this dose for 4 weeks; and
then increasing the dose to 3 mg QD, and administering at this dose as a maintenance dose for at least 4 weeks.

In one embodiment of the use of the free acid or hemicalcium salt of Compound 1a described above, oral administration is for a total treatment period selected from at least 40 weeks, at least 52 weeks, at least 72 weeks, and at least 104 weeks; wherein the total treatment period comprises dose escalation period and dose maintenance period.

In one embodiment of the use of the free acid or hemicalcium salt of Compound 1a described above, the oral administration is an adjunct to diet and exercise.

In one embodiment of the use or unit dosage having a maintenance dose of 3 mg QD, 6 mg QD, 12 mg QD, or 36 mg QD as described above, the subject is not restricted from food or water intake within 30 minutes before or after the oral administration of the free acid or the hemicalcium salt of Compound 1a.

In one embodiment of the use or unit dosage having a maintenance dose of 3 mg QD, 6 mg QD, 12 mg QD, or 36 mg QD as described above, the administration of the escalation doses results in less treatment related side effects as compared to a treatment at the respective maintenance doses without an escalation dose.

In one embodiment, a pharmaceutical composition is prepared as a dosage form, wherein the dosage form comprises between 1 mg and 45 mg, of Compound 1 having formula:

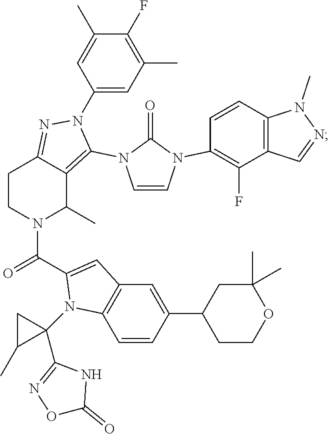 [SMILES: Cc1cc(-n2nc3c(c2-n2ccn(-c4ccc5c(cnn5C)c4F)c2=O)C(C)N(C(=O)c2cc4cc(C5CCOC(C)(C)C5)ccc4n2C2(c4noc(=O)[nH]4)CC2C)CC3)cc(C)c1F]

or a pharmaceutically acceptable salt thereof, or a hydrate of the compound or the pharmaceutically acceptable salt; and wherein the unit dosage form is suitable for oral administration.

In one embodiment, the method, the unit dosage, or the use as described herein is for treating a subject with T2D mellitus for improved glycemic control. In one embodiment, the subject is naïve to an insulin treatment prior to being treated with the compound. In one embodiment, the subject is already taking metformin prior to being treated with the compound. In one embodiment, the subject is on a background of metformin, SGLT-2, and/or sulfonylurea treatment and is at increased cardiovascular risk. In one embodiment, the subject is with T2D mellitus and inadequate glycemic control with diet and exercise alone or in combination with an oral antihyperglycemic medication. In one embodiment, the subject has T2D and is on a background treatment of titrated insulin glargine with or without metformin and/or SGLT-2.

In one embodiment, the method, the unit dosage, or the use as described herein is for treating a subject who is overweight and has a BMI (body mass index) of ≥27 kg/m2. In one embodiment, the subject has T2D and a BMI (body mass index) of ≥27 kg/m2. In one embodiment, the subject has a weight related comorbidity other than T2D and a BMI (body mass index) of ≥27 kg/m2. In one embodiment, a suitable subject has obesity and a BMI of ≥30 kg/m2. In one embodiment, a suitable subject has T2D and obesity with a BMI of ≥30 kg/m2.

In one embodiment, the method, the unit dosage, or the use as described herein is for treating a subject with T2D and with an HbA1c level of between ≥7.0% to ≤9.5%. In one embodiment, the HbA1c level of the subject is reduced by at least 1.0% after 40 weeks of treatment period following a Compound 1 or Compound 1a dosing regimen as described herein, wherein the treatment period of 40 weeks comprises both dose escalation and dose maintenance periods. In one embodiment, the HbA1c level of the subject is reduced by at least 1.5%. In one embodiment, the HbA1c level of the subject is reduced by at least 2.0%. In one embodiment, the HbA1c level of a subject is reduced to <7.0% after 40 weeks of treatment following a Compound 1 or Compound 1a dosing regimen. In one embodiment, a T2D treatment following a dosing regimen disclosed herein is superior to a comparable treatment with a placebo. In one embodiment, a higher percentage of subjects treated with a dosing regimen achieves an HbA1c level of <7.0% relative to a comparable placebo group. In one embodiment, the treatment also results in a weight reduction of at least 5% after 40 weeks of treatment period comprising dose escalation and dose maintenance periods. In one embodiment, the weight reduction is at least 10%. In one embodiment, the weight reduction is at least 15%.

In one embodiment, the method, the unit dosage, or the use as described herein is for treating a subject with T2D and has obesity or is overweight with increased cardiovascular risk. In one embodiment, a treatment following a Compound 1 or Compound 1a dosing regimen described herein is non-inferior to insulin glargine QD treatment (with a non-inferiority margin of 1.8) in the occurrence of MACE-4 events after 52 weeks of treatment period, wherein the treatment period of 52 weeks comprises both escalation and maintenance periods. In one embodiment, the treatment is superior to insulin glargine QD treatment in the occurrence of MACE-4 events. In one embodiment, the treatment also results in a weight reduction of at least 5% after 52 weeks of treatment period comprising dose escalation and dose maintenance periods. In one embodiment, the weight reduction is at least 10% after 52 weeks.

In one embodiment, the method, the unit dosage, or the use as described herein is for treating a subject with obesity or overweight with at least one weight related comorbidity. In one embodiment, the subject has obesity without T2D. In one embodiment, the subject has obesity with T2D. In one embodiment, the subject is overweight with at least one weight related comorbidity selected from hypertension, dyslipidemia, MASLD/MASH, cardiovascular disease, obstructive sleep apnea, osteoarthritis, prediabetes, increased risk for cancer, and increased risk for premature death. In one embodiment, the subject is overweight with T2D.

In one embodiment, the method, the unit dosage, or the use as described herein is for treating a subject who has obesity with T2D or is overweight with T2D, and a treatment following a Compound 1 or Compound 1a dosing regimen described herein results in a weight reduction of at least 5% after 72 weeks of treatment period, wherein the treatment period of 72 weeks comprises both dose escalation and dose maintenance periods. In one embodiment, the weight reduction is at least 10%. In one embodiment, the weight reduction is at least 15%.

In one embodiment, the method, the unit dosage, or the use as described herein is for treating a subject who has obesity without T2D or is overweight with at least one weight related comorbidity selected from cardiovascular disease, obstructive sleep apnea, osteoarthritis, increased risk for cancer, and increased risk for premature death. In one embodiment, a treatment following a Compound 1 or Compound 1a dosing regimen described herein results in a weight reduction of at least 5% after 72 weeks of treatment period, wherein the treatment period of 72 weeks comprises both dose escalation and dose maintenance periods. In one embodiment, the weight reduction is at least 10%. In one embodiment, the weight reduction is at least 15%.

In one embodiment, the Compound 1 or 1a dosing regimens, the uses of Compound 1 or 1a, and the pharmaceutical dosage forms comprising Compound 1 or 1a disclosed herein can be used to treat a subject with obstructive sleep apnea (OSA). OSA has been associated with obesity or overweight and the treatment of obesity or overweight as disclosed herein can additionally improve a subject's OSA condition.

In one embodiment of the method, the unit dosage form, or the use described herein, for each of the dosing schedule having a maintenance dose of 3 mg QD, 6 mg QD, 12 mg QD, and 36 mg QD, the total treatment period comprising dose escalation and dose maintenance is at least 40 weeks. In one embodiment, the total treatment period is at least 52 weeks. In one embodiment, the total treatment period is at least 72 weeks. In one embodiment, the total treatment period is at least 104 weeks.

In one embodiment, a method for treating an adult subject with type 2 diabetes and obesity or overweight at increased cardiovascular risk comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, for 4 weeks at this dose, if this dose is tolerated,
then increasing the dose to 6 mg QD, for 4 weeks at this dose, if this dose is tolerated,
then increasing the dose to 12 mg QD, for 4 weeks at this dose, if this dose is tolerated,
then increasing the dose to 24 mg QD, for 4 weeks at this dose, and if this dose is tolerated,
then increasing the dose to 36 mg QD, as a maintenance dose;
wherein if any of 3 mg QD, 6 mg QD, 12 mg QD, 24 mg QD is not tolerated, then stop at the maximum tolerated dose (MTD) and stay at the MTD dose as the maintenance dose;
wherein the maintenance dose is administered to the subject for at least 32 weeks or at least until the occurrence of about 122 MACE-4 events; and
wherein the treatment with Compound 1a is noninferior to an insulin glargine treatment (with a NIM of 1.8) in the occurrence of MACE-4. In one embodiment, the treatment with Compound 1a is superior to an insulin glargine treatment (with a NIM of 1.8) in the occurrence of MACE-4. In the embodiment, the total treatment period is at least 52 weeks which comprises dose escalation and dose maintenance. In the embodiment, the total treatment period is at least 104 weeks which comprises dose escalation and dose maintenance. In one embodiment, the treatment is an adjunct to diet and exercise.

In one embodiment, a method for treating an adult subject with type 2 diabetes and obesity or overweight at increased cardiovascular risk comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, for 4 weeks at this dose;
then increasing the dose to 6 mg QD, for 4 weeks at this dose;
then increasing the dose to 12 mg QD, for 4 weeks at this dose;
then increasing the dose to 24 mg QD, for 4 weeks at this dose; and
then increasing the dose to 36 mg QD, as a maintenance dose;
wherein the maintenance dose is administered to the subject for at least 32 weeks or until about 122 MACE-4 events have accrued; and
wherein the treatment with Compound 1a is noninferior to an insulin glargine treatment (with a NIM of 1.8) in the occurrence of MACE-4. In one embodiment, the treatment with Compound 1a is superior to an insulin glargine QD treatment (with a NIM of 1.8) in the occurrence of MACE-4. In one embodiment, the total treatment period is at least 52 weeks which comprises dose escalation and dose maintenance. In the embodiment, the total treatment period is at least 104 weeks which comprises dose escalation and dose maintenance. In one embodiment, the treatment is an adjunct to diet and exercise.

In one embodiment, a method for treating an adult subject with type 2 diabetes and inadequate glycemic control with diet and exercise alone comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 20 weeks;
wherein the HbA1c level of the subject is reduced to lower than 7% after 40 weeks of treatment period which comprises 20 weeks of dose escalation and 20 weeks of dose maintenance periods. In one embodiment, the HbA1c level of the subject is reduced to lower than 6.5% after 40 weeks of treatment.

In one embodiment, a method for treating an adult subject with type 2 diabetes and inadequate glycemic control with diet and exercise alone comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 28 weeks;
wherein the HbA1c level of the subject is reduced to lower than 7% after 40 weeks of treatment period which comprises 12 weeks of dose escalation and 28 weeks of dose maintenance periods. In one embodiment, the HbA1c level of the subject is reduced to lower than 6.5% after 40 weeks of treatment period.

In one embodiment, a method for treating an adult subject with type 2 diabetes and inadequate glycemic control with diet and exercise alone comprises:
orally administering to the subject a hemicalcium salt of Compound 1a or a hydrate thereof according to the following dosing schedule:
starting at a dose of 1 mg QD, and administering at this dose for 4 weeks; and
then increasing the dose to 3 mg QD, and administering at this dose as a maintenance dose for 36 weeks;
wherein the HbA1c level of the subject is reduced to lower than 7% after 40 weeks of treatment period which comprises 4 weeks of dose escalation and 36 weeks of dose maintenance periods. In one embodiment, the HbA1c level of the subject is reduced to lower than 6.5% after 40 weeks of treatment period.

In one embodiment, a method for treating an adult subject with type 2 diabetes and inadequate glycemic control with metformin comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 20 weeks;
wherein the HbA1c level of the subject is reduced to lower than 7% after 40 weeks of treatment period which comprises 20 weeks of dose escalation and 20 weeks of dose maintenance periods. In one embodiment, the HbA1c level of the subject is reduced to lower than 6.5% after 40 weeks of treatment period. In one embodiment, the treatment with Compound 1a after the 36 mg maintenance dose is noninferior to a 10 mg QD oral dapagliflozin treatment in glycemic control.

In one embodiment, a method for treating an adult subject with type 2 diabetes and inadequate glycemic control with metformin comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 28 weeks;
wherein the HbA1c level of the subject is reduced to lower than 7% after 40 weeks of treatment period which comprises 12 weeks of dose escalation and 28 weeks of dose maintenance periods. In one embodiment, the HbA1c level of the subject is reduced to lower than 6.5% after 40 weeks of treatment. In one embodiment, the treatment with Compound 1a after the 12 mg maintenance dose is noninferior to a 10 mg QD oral dapagliflozin treatment in glycemic control.

In one embodiment, a method for treating an adult subject with type 2 diabetes and inadequate glycemic control with metformin comprises:
orally administering to the subject a hemicalcium salt of Compound 1a or a hydrate thereof according to the following dosing schedule:
starting at a dose of 1 mg QD, and administering at this dose for 4 weeks; and
then increasing the dose to 3 mg QD, and administering at this dose as a maintenance dose for at least 36 weeks;
wherein the HbA1c level of the subject is reduced to lower than 7% after 40 weeks of treatment period which comprises 4 weeks of dose escalation and 36 weeks of dose maintenance periods. In one embodiment, the HbA1c level of the subject is reduced to lower than 6.5% after 40 weeks of treatment period. In one embodiment, the treatment with Compound 1a after the 3 mg maintenance dose is noninferior to a 10 mg QD oral dapagliflozin treatment in glycemic control.

In one embodiment, a method for treating an adult subject with type 2 diabetes and inadequate glycemic control with metformin comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 32 weeks;
wherein the HbA1c level of the subject is reduced to lower than 7% after 52 weeks of treatment period which comprises 20 weeks of dose escalation and 32 weeks of dose maintenance periods; and
wherein the treatment with Compound 1a is noninferior to a 7 mg QD semaglutide treatment (with a 3 mg QD semaglutide escalation for 4 weeks) in glycemic control. In one embodiment, the treatment with Compound 1a is noninferior to a 14 mg QD semaglutide treatment (with successive 3 mg QD and 7 mg QD semaglutide escalations for 4 weeks each) in glycemic control.

In one embodiment, a method for treating an adult subject with type 2 diabetes and inadequate glycemic control with metformin comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 40 weeks;
wherein the HbA1c level of the subject is reduced to lower than 7% after 52 weeks of treatment period which comprises 12 weeks of dose escalation and 40 weeks of dose maintenance periods; and
wherein the treatment with Compound 1a is noninferior to a 7 mg QD semaglutide treatment (with an initial 3 mg QD semaglutide escalation for 4 weeks) in glycemic control. In one embodiment, the treatment with Compound 1a is noninferior to a 14 mg QD semaglutide treatment (with initial successive 3 mg QD for 4 weeks and 7 mg QD for 4 weeks of semaglutide escalations) in glycemic control.

In one embodiment, a method for treating an adult subject with type 2 diabetes and inadequate glycemic control with insulin glargine, and with or without metformin and/or SGLT-2, comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 20 weeks;
wherein the HbA1c level of the subject is reduced to lower than 7% after 40 weeks of treatment period which comprises 20 weeks of dose escalation and 20 weeks of dose maintenance periods. In one embodiment, the HbA1c level of the subject is reduced to lower than 6.5% after 40 weeks of treatment period. In one embodiment, the treatment with Compound 1a is superior to placebo in glycemic control.

In one embodiment, a method for treating an adult subject with type 2 diabetes and inadequate glycemic control with insulin glargine, and with or without metformin and/or SGLT-2, comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 28 weeks;
wherein the HbA1c level of the subject is reduced to lower than 7% after 40 weeks of treatment period which comprises 12 weeks of dose escalation and 28 weeks of dose maintenance periods. In one embodiment, the HbA1c level of the subject is reduced to lower than 6.5% after 40 weeks of treatment period. In one embodiment, the treatment with Compound 1a is superior to placebo in glycemic control.

In one embodiment, a method for treating an adult subject with type 2 diabetes and inadequate glycemic control with insulin glargine, and with or without metformin and/or SGLT-2, comprises:
orally administering to the subject a hemicalcium salt of Compound 1a or a hydrate thereof according to the following dosing schedule:
starting at a dose of 1 mg QD, and administering at this dose for 4 weeks; and
then increasing the dose to 3 mg QD, and administering at this dose as a maintenance dose for at least 36 weeks;
wherein the HbA1c level of the subject is reduced to lower than 7% after 40 weeks of treatment period which comprises 4 weeks of dose escalation and 36 weeks of dose maintenance periods. In one embodiment, the HbA1c level of the subject is reduced to lower than 6.5% after 40 weeks of treatment period. In one embodiment, the treatment with Compound 1a is superior to placebo in glycemic control.

In one embodiment, a method for treating an adult subject with obesity or overweight with at least one weight related comorbidity other than T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 52 weeks;
wherein the oral administration of Compound 1a results in a weight reduction of at least 5% after 72 weeks of treatment period which comprises 20 weeks of dose escalation and 52 weeks of dose maintenance periods. In one embodiment, weight reduction is at least 10%. In one embodiment, weight reduction is at least 15%. In one embodiment, weight reduction is at least 20%.

In one embodiment, a method for treating an adult subject with obesity or overweight with at least one weight related comorbidity other than T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 60 weeks;
wherein the oral administration of Compound 1a results in a weight reduction of at least 5% after 72 weeks of treatment period which comprises 12 weeks of dose escalation and 60 weeks of dose maintenance periods. In one embodiment, the weight reduction is at least 10%. In one embodiment, the weight reduction is at least 15%. In one embodiment, the weight reduction is at least 20%.

In one embodiment, a method for treating an adult subject with obesity or overweight with at least one weight related comorbidity other than T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 64 weeks;
wherein the oral administration of Compound 1a results in a weight reduction of at least 5% after 72 weeks of treatment period which comprises 8 weeks of dose escalation and 64 weeks of dose maintenance periods. In one embodiment, the weight reduction is at least 10%. In one embodiment, the weight reduction is at least 15%.

In one embodiment, a method for treating an adult subject with obesity or overweight with T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 52 weeks;
wherein the oral administration of Compound 1a results in a weight reduction of at least 5% after 72 weeks of treatment period which comprises 20 weeks of dose escalation and 52 weeks of dose maintenance periods. In one embodiment, weight reduction is at least 10%. In one embodiment, weight reduction is at least 15%. In one embodiment, the oral administration of Compound 1a additionally results in a reduction of the subject's HbA1c level by at least 1.0%. In one embodiment, the reduction of the HbA1c level is at least 1.2%. In one embodiment, the reduction of the HbA1c level is at least 1.5%. In one embodiment, the subject's HbA1c level is reduced to lower than 7.0% after 72 weeks of treatment period. In one embodiment, the subject's HbA1c level is reduced to lower than 6.5% after 72 weeks of treatment period.

In one embodiment, a method for treating an adult subject with obesity or overweight with T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 60 weeks;
wherein the oral administration of Compound 1a results in a weight reduction of at least 5% after 72 weeks of treatment period which comprises 12 weeks of dose escalation and 60 weeks of dose maintenance periods. In one embodiment, weight reduction is at least 10%. In one embodiment, weight reduction is at least 15%. In one embodiment, the oral administration of Compound 1a additionally results in a reduction of the subject's HbA1c level by at least 1.0%. In one embodiment, the reduction of the HbA1c level is at least 1.2%. In one embodiment, the reduction of the HbA1c level is at least 1.5%. In one embodiment, the subject's HbA1c level is reduced to lower than 7.0% after 72 weeks of treatment period. In one embodiment, the subject's HbA1c level is reduced to lower than 6.5% after 72 weeks of treatment period.

In one embodiment, a method for treating an adult subject with obesity or overweight with T2D comprises:
orally administering to the subject a hemicalcium salt of Compound 1a or a hydrate thereof according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 64 weeks;
wherein the oral administration of Compound 1a results in a weight reduction of at least 5% after 72 weeks of treatment period which comprises 8 weeks of dose escalation and 64 weeks of dose maintenance periods. In one embodiment, weight reduction is at least 10%. In one embodiment, weight reduction is at least 15%. In one embodiment, the oral administration of Compound 1a additionally results in a reduction of the subject's HbA1c level by at least 1.0%. In one embodiment, the reduction of the HbA1c level is at least 1.2%. In one embodiment, the reduction of the HbA1c level is at least 1.5%. In one embodiment, the subject's HbA1c level is reduced to lower than 7.0% after 72 weeks of treatment period. In one embodiment, the subject's HbA1c level is reduced to lower than 6.5% after 72 weeks of treatment period.

In one embodiment, a hemicalcium salt of Compound 1a or a hydrate thereof is used for the manufacture of a medicament for treating a subject with T2D, obesity, or overweight with at least one weight related comorbidity according to the dosing schedules as described in the above embodiments.

In one embodiment, a treatment period is preceded by a screening or lead-in period. In one embodiment, a treatment period is followed by an additional treatment or a safety follow-up period.

As used herein, an “effective amount” or “effective dose” of a GLP-1 receptor NPA means an amount sufficient to cure, alleviate, or partially arrest the clinical manifestations of a given disease or state and its complications. An amount adequate to accomplish this is defined as “effective amount” or “effective dose”. The effective amount for each purpose will depend on the severity of the disease or condition as well as the weight and general state of the subject.

As used herein, the term “maximum tolerated dose” or “MTD” means the highest dose of a drug or treatment that does not cause unacceptable side effects. In one embodiment, the maximum tolerated dose is determined in clinical trials by testing increasing doses on different groups of people until the highest dose with acceptable side effects is found.

As used herein, the term “treatment” or “treating” means the management and care of a subject for the purpose of combating a condition, such as a disease or a disorder. In one embodiment, the term “treatment” or “treating” is intended to include the full spectrum of treatments for a given condition from which the subject is suffering, such as administration of an active GLP-1 receptor NPA to alleviate the symptoms or complications; to delay the progression of a disease, disorder, or condition; or to cure or eliminate the disease, disorder, or condition.

As used herein, the terms “Compound LY” and “Compound 1” are used interchangeably.

As used herein, the term “OFG” means a hemicalcium salt of Compound 1a or a hydrate thereof, as is clear from the context.

As used herein, the terms “dose escalation” and “escalation dose” are used interchangeably with “dose titration” and “titration dose”, respectively. Similarly, dose “escalation” and “titration” are used interchangeably.

As used herein, the terms “escalating” and “increasing” when referring to changing a dose are used interchangeably.

As used herein, the terms “dosing schedule” and “dosing regimen” are used interchangeably.

As used herein, the term “at the start of the treatment” means the time point of the start of a dose escalation or treatment after a screening/lead-in period. The weight or HbA1C level of a subject at the start of a treatment is sometimes referred to as the baseline weight or HbA1C level of the subject.

As used herein, “weight loss” and “weight reduction” are used interchangeably.

### EXAMPLES

### Capsule Formulation of Compound 1 (0.5 Ca Hydrate)

In one embodiment, Compound 1 is administered to a subject in a capsule. In one embodiment, a capsule comprising Compound 1 can be prepared as described in Examples 1 and 2 below.

### Example 1

### Compound 1a 0.5 Ca Hydrate SDD Preparation

Compound 1a 0.5 Ca hydrate was dissolved in ethanol, denatured with methanol (5% v/v or w/w). A 20% w/w solids solution was prepared with 30% w/w of the solid fraction composed of the title compound (on a free acid basis) and the balance composed of PVP-VA. This translated to 6% of Compound 1a (on a free acid basis), 14% PVP-VA and 80% of denatured ethanol SDA-3A—all fractions as w/w. After spray drying, the solids that formed were composed of 30% w/w of the solid fraction composed of Compound 1a (on a free acid basis) and the balance composed of PVP-VA. The % values are shown in Table 1 below.

### Example 1—Compound 1a SDD

TABLE 1
Compound 1a SDD Compositions

| | Solution | Amorphous SDD |
|---|---|---|
| | (mass fractions) | (mass fraction) |
| Compound 1a | 6% | 30.0% |
| PVP-VA | 14% | 70.0% |
| EtOH SDA-3A | 80% | |
| EtOH | 76% | |
| MeOH | 4% | |

Once solution was prepared, the solution was pumped to a spray dryer where the solution atomizes upon entry. Heated drying gas entered co-current to the atomized liquid at the top of the spray drying chamber at an approximate ratio of 0.044 kg/kg of spray solution to drying gas. The inlet temperature was adjusted to provide an outlet temperature of 35 to 45° C. The solids formed in the spray dryer were collected from a cyclone as well as a filter housing on the gas stream. The gas was passed over a condenser maintained at −3° C. to remove (to a dewpoint of −3° C.) solvent. The gas was then heated to the inlet temperature and passed back to the spray dryer.

### Example 2—Exemplary Capsule Formulation

### Compound 1a 0.5 Ca Hydrate, 1 mg and 15 mg Capsule Formulation

The SDD, NaHCO₃, microcrystalline cellulose (MCC PH-102), and SiO2 were dispensed into separate low-density polyethylene (LDPE) bags. NaHCO₃ and MCC PH-102 were passed through a screen (30 mesh) sequentially into separate LDPE bags. ~25% of the sieved NaHCO₃ was added to a 10 L mixing bin. Sieve MCC was added to the bag containing the SDD and manually mixed for at least two minutes. Then SiO2 was added to the MCC and SDD blend and manually mixed for at least two more minutes. The blend was sieved through a 30 mesh screen into the bin. The blend LDPE bag was rinsed with the remaining NaHCO₃, sieved through a 30 mesh screen into the bin, then blended for 15 minutes at 20 RPM. The blend was sieved through a 30 mesh screen and blended again for 15 min at 20 RPM. The blend was filled into empty 0 capsule shells to the target fill weight using a semi-automated filling machine (e.g. Dott-Bonapace).

TABLE 2
Capsule 1 mg and 15 mg Compositions

| | | Ex 3 capsule, | Ex 3 capsule, |
|---|---|---|---|
| | Function | 1 mg, mg/capsule | 15 mg, mg/capsule |
| Ex. 1 SDD | | 3.36 | 50.34 |
| NaHCO₃ | pH modifier | 200.0 | 200.0 |
| MCC PH-102 | filler/binder | 44.14 | 7.06 |
| SiO₂, SYLOID | glidant | 2.5 | 2.6 |
| 244 FP (1%) | | | |
| Total (mg) | | 250.0 | 260.0 |

### Tablet and Capsule Formulations of Compound 1a 0.5 Ca Hydrate

In one embodiment, Compound 1a is administered to a subject in a tablet. In one embodiment, exemplary tablets comprising Compound 1a can be prepared as described in the following Examples.

### Example 3 Exemplary Tablets and Alternative Capsules

### Compound 1a SDD Preparation 1

30% Compound 1a SDD was prepared by dissolving 3-[(1S,2S)-1-[5-[(4S)-2,2-dimethyloxan-4-yl]-2-[(4S)-2-(4-fluoro-3,5-dimethylphenyl)-3-[3-(4-fluoro-1-methylindazol-5-yl)-2-oxoimidazol-1-yl]-4-methyl-6,7-dihydro-4H-pyrazolo[4,3-c]pyridine-5-carbonyl]indol-1-yl]-2-methylcyclopropyl]-4H-1,2,4-oxadiazol-5-one, 0.5 Ca hydrate (8.7 g, 92% potency) and PVP-VA, (also known as copovidone, CAS #25086-89-9;) (18.7 g) in EtOH (200 mL) at room temperature. Following dissolution of the solids the solution was spray dried on a conventional spray dryer with a pressure nozzle. The following parameters in the following Table were used to create the dispersion using a laboratory scale spray drier. Spray drying began when the spray dryer temperature is above 33° C. The material was collected and dried under vacuum at 50° C. overnight to give the title compound (21.99 g, 7.0 g, 92% potency, 80% recovery) and observed via photomicroscopy that the material was microscopically non birefringent particles that were approximately 5-25 μm in diameter.

TABLE 3
SDD Parameters

| Process Parameter | Value |
|---|---|
| Aspirator | 100% |
| Nebulization Flow | 37 mm (~400 L/hr) |
| Solution flow rate | 40% (12.5 mL/min) |
| Chiller Temperature | −20° C. |
| Drying Gas Inlet | 120° C. |
| Temperature | |
| Spray Dryer Outlet | 75° C. |
| Temperature (° C.) | |

### Compound 1a SDD Preparation 2

Compound 1a was dissolved in EtOH, denatured with MeOH (5% v/v or w/w). A 20% w/w solids solution was prepared with 30% w/w of the solid fraction composed of the title compound; (on a free acid basis) and the balance composed of PVP-VA. This translated to 6% of the title compound (on a free acid basis), 14% PVP-VA and 80% of denatured EtOH SDA-3A—all fractions as w/w. After spray drying, the solids that form were composed of 30% w/w of the solid fraction composed of the title compound (on a free acid basis) and the balance composed of PVP-VA. The % values are shown in the Table below.

TABLE 4
Formulation of Preparation 2 SDD

| | Solution | Amorphous SDD |
|---|---|---|
| | (mass fractions) | (mass fraction) |
| Title compound | 6% | 30.0% |
| PVP-VA | 14% | 70.0% |
| EtOH SDA-3A | 80% | |
| EtOH | 76% | |
| MeOH | 4% | |

Once the solution was prepared, the solution was pumped to a spray dryer where the solution atomized upon entry. Heated drying gas entered co-current to the atomized liquid at the top of the spray drying chamber at an approximate ratio of 0.044 kg/kg of spray solution to drying gas. The inlet temperature was adjusted to provide an outlet temperature of 35 to 45° C. The solids formed in the spray dryer were collected from a cyclone as well as a filter housing on the gas stream. The gas was passed over a condenser maintained at −3° C., to remove (to a dewpoint of −3° C.) solvent. The gas was then heated to the inlet temperature and passed back to the spray dryer.

### Preparation 3

### Compound 1a Capsule Preparation

Capsules were prepared by first adding sodium bicarbonate (600 mg) to a size 0 hypromellose capsule followed by Preparation 3 SDD (54 mg)

TABLE 5
Quantitative Capsule Composition, Preparation 3

| Capsule | Wt % | mg/tablet |
|---|---|---|
| Preparation 3 SDD | 8.26 | 54 |
| Sodium bicarbonate | 91.74 | 600 |
| (anhydrous) | | |
| Total | 100 | 654 |

### Compound 1a Tablet Formulation

### Core Tablet Composition Preparation

To prepare core tablets, Compound 1a (13.33% w/w) SDD and excipients, (as set forth below), except magnesium stearate, were screened through a 600 μm sieve before use. The materials were added to a vessel (1000 mL) and mixed with a mixer at 32 rpm for about 10 min. The mixture blend was passed through sieve (600 μm) and mixed with mixer a 2nd time at 32 rpm for about 10 min. Magnesium stearate was added and the mixture was mixed at 32 rpm for about 3 min. The mixture blend was compressed using a 14.10×7.75 mm oval tablet tooling on a single station tablet press to produce tablets (400.0 mg) with a hardness of 30 kP.

Tablet compositions were prepared substantially as described herein above, using parameters of the following Tables to prepare corresponding Examples (Compositions T1, T2, A, B, C, D, and E). Each example included an IR coating substantially as described herein and additionally included an enteric coating. A tablet of Example 7E included only an IR coating.

TABLE 6
Tablet composition T1

| | | Composition T1 | | |
|---|---|---|---|---|
| | | | | Quantity/300 |
| Component | Function | % w/w | mg/tablet | g batch (g) |
| Prep 2 SDD | Drug | 13.33 | 53.33 *(16.00) | 39.48 (39.99) |
| (30 wt %) | | | | |
| Micro- | Filler | 56.67 | 226.67 | 170.52 (170.01) |
| crystalline | | | | |
| cellulose | | | | |
| Na₂CO₃ | pH | 10.00 | 40.00 | 30.00 |
| anhydrous | modifier | | | |
| Crospo- | disintegrant | 17.50 | 70.00 | 52.50 |
| vidone | | | | |
| XL-10 | | | | |
| Silicon | Glidant | 2.00 | 8.00 | 6.00 |
| dioxide | | | | |
| Magnesium | Lubricant | 0.50 | 2.00 | 1.50 |
| stearate | | | | |
| Total | | 100.00 | 400.00 | 300.00 |

TABLE 7
Tablet Composition T2

| | | Composition T2 | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 39.99 |
| (30 wt %) | | | | |
| Microcrystalline | Filler | 72.17 | 288.67 | 216.51 |
| cellulose | | | | |
| Na₂CO₃ | pH | 10.00 | 40.00 | 30.00 |
| anhydrous | modifier | | | |
| Crospovidone | disintegrant | 2.00 | 8.00 | 6.00 |
| XL-10 | | | | |
| Silicon | Glidant | 2.00 | 8.00 | 6.00 |
| dioxide | | | | |
| Magnesium | lubricant | 0.50 | 2.00 | 1.50 |
| stearate | | | | |
| Total | | 100.00 | 400.00 | 300.00 |

TABLE 8
Tablet Composition A

| | | Composition A | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 38.86 |
| (30 wt %) | | | | |
| Microcrystalline | Filler | 61.67 | 246.67 | 186.14 |
| cellulose | | | | |
| Na₂CO₃ | pH | 5.00 | 20.00 | 15.00 |
| anhydrous | modifier | | | |
| Crospovidone | disintegrant | 17.50 | 70.00 | 52.50 |
| XL-10 | | | | |
| Silicon | Glidant | 2.00 | 8.00 | 6.00 |
| dioxide | | | | |
| Magnesium | lubricant | 0.50 | 2.00 | 1.50 |
| stearate | | | | |
| Total | | 100.00 | 400.00 | 300.00 |

TABLE 9
Tablet Composition B

| | | Composition B | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 38.86 |
| (30 wt %) | | | | |
| Microcrystalline | Filler | 51.67 | 206.67 | 156.14 |
| cellulose | | | | |
| Na₂CO₃ | pH | 15.00 | 60.00 | 45.00 |
| anhydrous | modifier | | | |
| Crospovidone | disintegrant | 17.50 | 70.00 | 52.50 |
| XL-10 | | | | |
| Silicon | Glidant | 2.00 | 8.00 | 6.00 |
| dioxide | | | | |
| Magnesium | Lubricant | 0.50 | 2.00 | 1.50 |
| stearate | | | | |
| Total | | 100.00 | 400.00 | 300.00 |

TABLE 10
Tablet Composition C

| | | Composition C | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 38.86 |
| (30 wt %) | | | | |
| Microcrystalline | Filler | 69.17 | 276.67 | 208.64 |
| cellulose | | | | |
| Na₂CO₃ | pH | 15.00 | 60.00 | 45.00 |
| anhydrous | modifier | | | |
| Silicon | Glidant | 2.00 | 8.00 | 6.00 |
| dioxide | | | | |
| Magnesium | Lubricant | 0.50 | 2.00 | 1.50 |
| stearate | | | | |
| Total | | 100.00 | 400.00 | 300.00 |

TABLE 11
Tablet Composition D

| | | Composition D | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 39.98 |
| (30 wt %) | | | | |
| Microcrystalline | Filler | 79.17 | 316.67 | 20.00 |
| cellulose | | | | |
| Na₂CO₃ | pH | 5.00 | 20.00 | 8.00 |
| anhydrous | modifier | | | |
| Silicon | Glidant | 2.00 | 8.00 | 2.00 |
| dioxide | | | | |
| Magnesium | Lubricant | 0.50 | 2.00 | 400.00 |
| stearate | | | | |
| Total | | 100.00 | 400.00 | 300.00 |

TABLE 12
Tablet Composition E

| | | Composition E | | |
|---|---|---|---|---|
| | | | | Quantity/300 |
| Component | Function | % w/w | mg/tablet | g batch (g) |
| Prep 2 SDD | Drug | 13.33 | 53.33 (16.00) | 38.86 |
| (30 wt %) | | | | |
| Microcrystalline | Filler | 56.67 | 226.67 | 171.14 |
| cellulose | | | | |
| Na₂CO₃ | pH | 10.00 | 40.00 | 30.00 |
| anhydrous | modifier | | | |
| Crospovidone | Disintegrant | 17.50 | 70.00 | 52.50 |
| XL-10 | | | | |
| Silicon | Glidant | 2.00 | 8.00 | 6.00 |
| dioxide | | | | |
| Magnesium | Lubricant | 0.50 | 2.00 | 1.50 |
| stearate | | | | |
| Total | | 100.00 | 400.00 | 300.00 |

### Example 4—Clinical Data

The clinical pharmacology, PK, and PD of Compound 1a were studied in 2 completed Phase 1 studies in healthy volunteers and in participants with T2D). Results from these studies demonstrated a PK profile appropriate for once daily oral dosing that can be administered without limitations pertaining to food or water intake or time of day. The PK of Compound 1a plasma concentration increase was approximately proportional to the increase of dose across the 9 mg QD to 45 mg QD dose range.

In these Phase 1 studies, GI AEs of nausea, vomiting, and constipation were the most reported AEs. PD results from one study showed clinically relevant improvements in HbA1c of up to −1.4%, and weight loss of up to −5.8 kg after 12 weeks of treatment with 9 mg of Compound 1a QD to 45 mg QD in people with T2D.

Two Phase 2 studies have evaluated the safety and efficacy of Compound 1a for the treatment of T2D and another for treatment of obesity or overweight with at least one weight related comorbidity. In one study, Compound 1a treatment for 26 weeks at doses of 3 mg to 45 mg QD resulted in mean changes from baseline in HbA1c up to −2.1% (treatment difference of −1.7% vs placebo) and mean weight loss of up to 10 kg (treatment difference of 8 kg vs placebo).

In another Phase 2 study in people with obesity or overweight with at least one weight related comorbidity, mean percent weight loss of up to 13% from baseline (11% compared to placebo) was observed with Compound 1a treatment at the primary 26-week endpoint. Most common TEAEs were GI related (nausea, vomiting, diarrhea and constipation).

The detailed results of these two Phase 2 studies are provided in more detail in Examples 4A and 4B below.

### Example 4A—Phase 2 Clinical Data

In a randomized, double-blind Phase 2 clinical trial, 272 adults were enrolled with obesity or overweight with at least one weight related comorbidity, excluding diabetes, to examine the weight reduction efficacy and safety of 4 dose levels (12 mg, 24 mg, 36 mg and 45 mg) of Compound 1a hemicalcium salt (“OFG”) compared with once daily administered placebo for 36 weeks. The primary endpoint was the percentage change in body weight from baseline to week 26, with a secondary 36-week endpoint.

### Trial Design

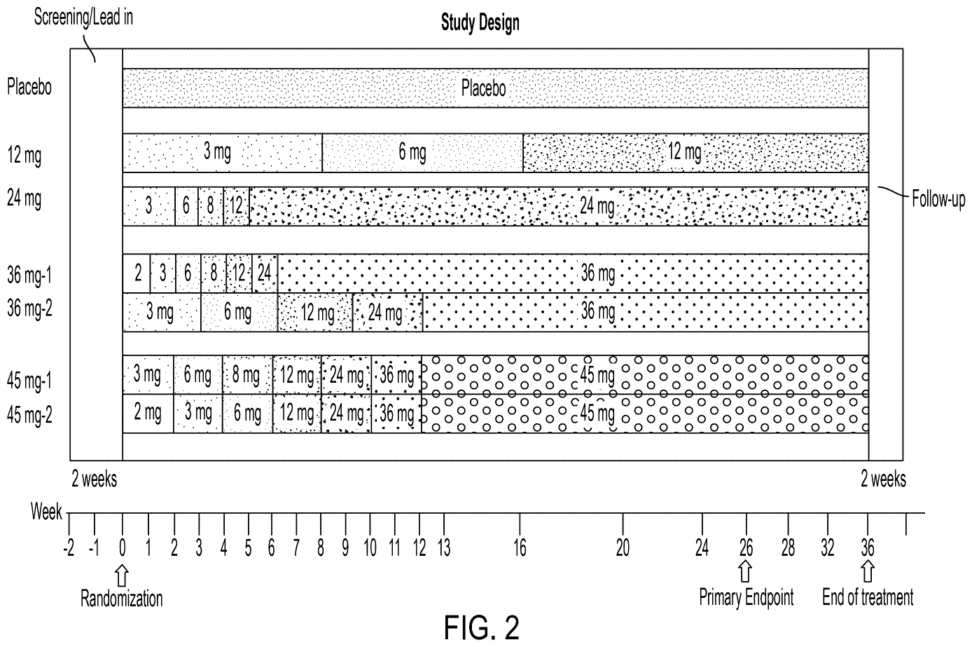

A 36-week Phase 2, multicenter, randomized, double-blind, parallel, placebo-controlled study was designed to examine the efficacy and safety of 4 dose levels of once daily administered Compound 1a, compared with once daily administered placebo in participants with obesity or overweight with at least one weight related comorbidity. A total of 272 patients were randomized 5:5:5:3:3:3:3 to once daily placebo or Compound 1a maintenance doses of 12 mg, 24 mg, 36 mg (36-1, 36-2) or 45 mg (45-1, 45-2) treatment groups. The 36 and 45 mg cohorts were subdivided in a 36 mg-1 and 36 mg-2 as well as 45 mg-1 and 45 mg-2 respectively, as different starting doses, dose escalations and dose escalation steps were investigated in these subgroup cohorts (FIG. 2). The different dose escalations were performed to assess the maintenance dose and titration steps for future trials.

### Participants

Eligible male or female participants were >18 years of age with a HbA1c<6.5% (48 mmol/mol) and a BMI of ≥30 kg/m2 or ≥27 kg/m2 and <30 kg/m2 with at least 1 of the following weight-related comorbidities: hypertension, dyslipidemia, cardiovascular disease, or obstructive sleep apnea. Participants had a stable body weight (<5% body weight gain and/or loss) for the 3 months prior to randomization.

### Procedures

The study period included a 2-week screening/lead in period, a 36-week treatment period followed by a 2 week off-drug safety follow-up period. During the treatment period, doses were escalated for all treatment groups. The dose escalation period ranged from 0 to 16 weeks depending on dose group. The initial dose was 2 or 3 mg followed by additional escalation steps depending on assigned cohort (FIG. 2). Compound 1a or matching placebo was administered daily by oral capsule. All participants were provided healthy eating and exercise education by study personnel throughout the trial.

### Endpoint

The primary endpoint was percent change in body weight from baseline at week 26. Secondary endpoints included percent change in body weight from baseline at week 36, change from baseline in body weight (kg), BMI (kg/m2) and waist circumference (cm), and percentage of study participants who achieved ≥5% body weight (kg) reduction, and ≥10% body weight (kg) reduction, at week 26 and week 36. Key safety endpoints included adverse events, blood pressure, heart rate, safety laboratory measures, PK parameters, and patient-reported outcomes.

### Statistical Analyses

A sample size of 270 participants was calculated to provide at least 90% power for testing superiority of Compound 1 versus placebo in the primary endpoint. The estimand used for efficacy analyses was an “efficacy estimand”, representing the average treatment effect for the randomized population if the treatment was administered as intended, thus including data from all randomized participants who received at least one dose of study drug and excluding data after permanent discontinuation of study drug. All tests of treatment effects were conducted at a 2-sided alpha level of 0.05 without adjustment for multiple comparisons, and 2-sided 95% CIs were calculated. To test efficacy of Compound 1a with adequate statistical power, for 36 mg and 45 mg the evaluation of the primary endpoint was made by pooling 2 dose escalation regimens (combining 36 mg-1 and 36 mg-2, for 36 mg, and combining 45 mg-1 and 45 mg-2, for 45 mg).

Safety analyses were conducted by comparing safety of Compound 1a with placebo irrespective of adherence to study drug. The safety analyses were conducted using the safety analysis set, which includes all randomized participants who received at least one dose of study drug. The results were presented in summary statistics, point estimates, and 95% confidence intervals along with p-values for treatment comparisons.

### Results

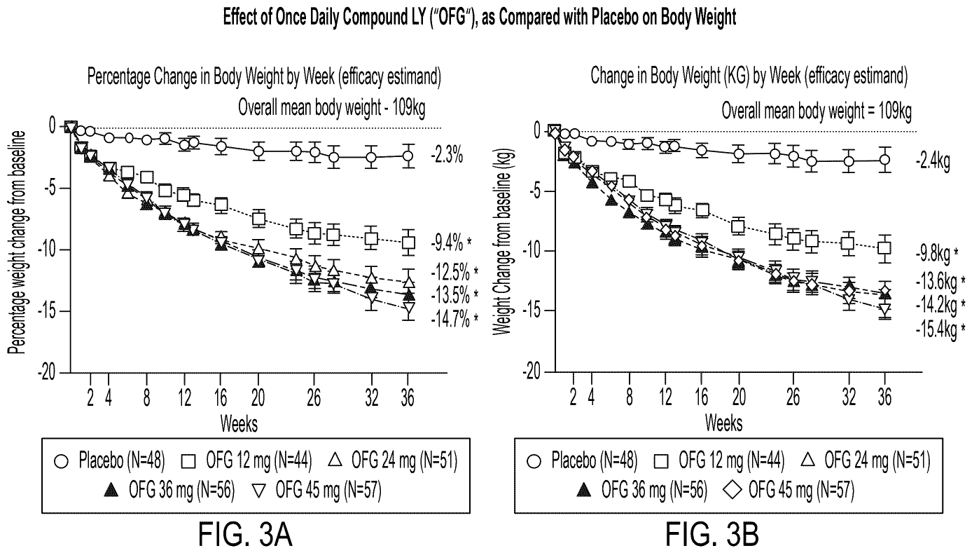

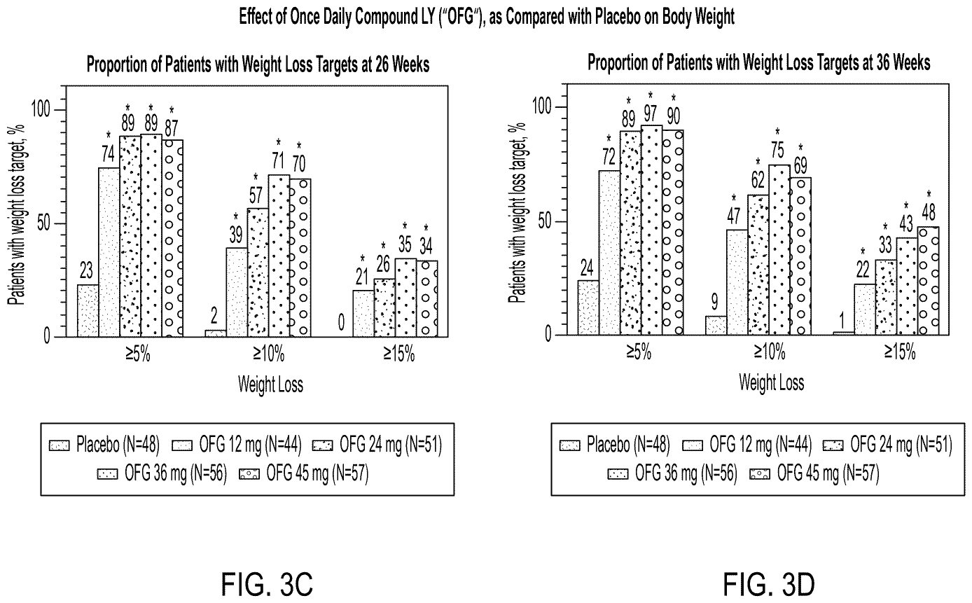

At baseline the mean body weight was 108.7 kg and the mean BMI was 37.9 kg/m2. The mean percentage change in body weight from baseline ranged from −8.6% to −12.6% at week 26, in a dose dependent manner, and −9.4% to −14.7% at week 36, compared with −2.0% and −2.3% with placebo respectively (FIG. 3). The percentage of patients who had weight reduction of 10% or more at 36 weeks ranged from 47% to 75%, versus 9% with placebo. At the highest Compound 1a dose (45 mg), 48% of patients had a weight reduction of 15% or more at 36 weeks compared with 1% for placebo. Treatment with Compound 1a resulted in improvements in all prespecified weight and cardiometabolic measures. The most common adverse events with Compound 1a were gastrointestinal, mild to moderate in severity, and occurred primarily during dose escalation. The tolerability and safety profile are consistent with the GLP-1 RA class.

Across all treatment groups there was a statistically significant, dose dependent, continuous decrease in placebo corrected body weight from baseline to 26- and 36-weeks, in all treatment groups, ranging from −6.9 kg to −11.2 kg (all p<0.001) at 26-weeks and −7.4 kg to −13.0 kg (all p<0.001) at 36-weeks (FIG. 3).

### Additional Treatment Outcomes

There was a mean reduction in systolic blood pressure (SBP) from baseline to week 26 (ranging from −4.8 to −10.5 mmHg) and from baseline to week 36 (ranging from −6.7 to −10.5 mmHg) in the Compound 1a groups, compared with −3.6 and −1.8 for placebo respectively. SBP tended to decrease in all Compound 1a groups relative to placebo during the study. No differences were noted in diastolic blood pressure (DBP) when compared to placebo. Benefits with Compound 1a were noted with respect to changes in the fasting lipids (triglycerides, total cholesterol, non-HDL cholesterol, LDL and VLDL) while there was no significant change in HDL cholesterol versus placebo at week 36. Participants who received Compound 1a treatment reported greater improvements in health-related quality of life and physical activity readouts versus placebo.

Changes to systolic or diastolic blood pressure and mean pulse rate compared with placebo were consistent with the results found in the Phase 2 study described in Example 4B. Prevalence, incidence, and onset of nausea, vomiting, diarrhea, and constipation observed in this study are also generally consistent with the results found in Example 4B.

### Tolerability and Safety

Nausea, constipation, vomiting, diarrhea, and eructation were the most common events reported with Compound 1a and were more frequent on Compound 1a vs. placebo. Most gastrointestinal events were mild-to-moderate in severity, occurred mainly during initial titrations and were transient, and resolved without permanent discontinuation of the treatment. Overall, the percentage of patients who reported AEs was slightly higher in the Compound 1a groups (86% to 90.2%) vs placebo (76.0%). The most frequent GI reactions did not increase in a dose-dependent manner with Compound 1a dose above 3 mg. The starting dose of 3 mg had higher GI AE events throughout the trial versus the 2 mg starting dose. There was a superior tolerability if dose was escalated every 3 weeks compared to every 1-2 weeks. Overall, after the titration phase, Compound 1a was well tolerated across all arms. Liver function tests were not significantly increased in participants receiving Compound 1a compared with placebo.

### Example 4B—Phase 2 Clinical Data

### Trial Design

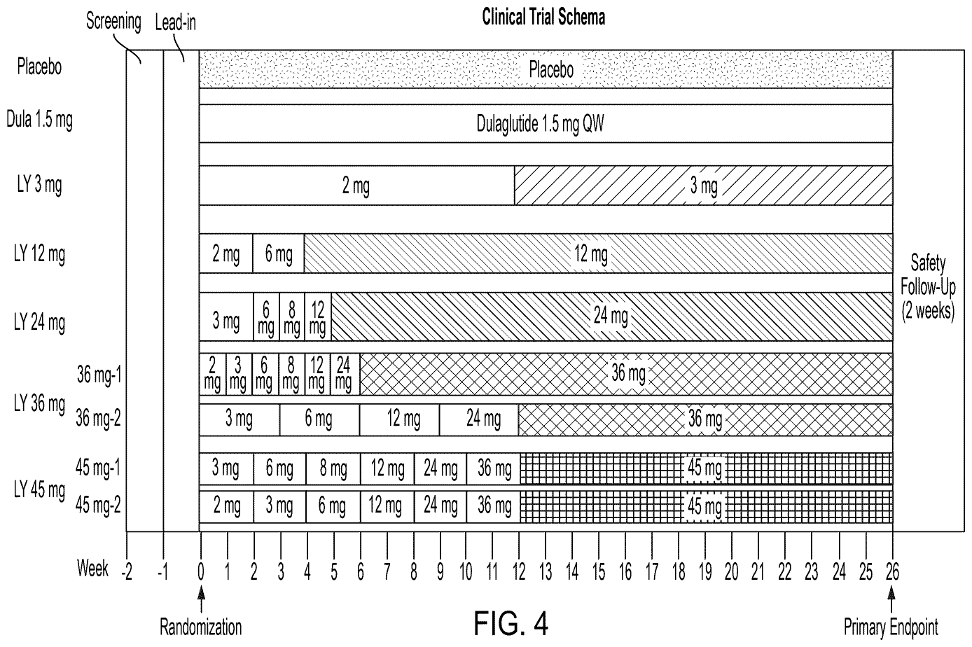

A 26-week Phase 2, multicenter, randomized, double-blind, parallel, placebo-controlled study was designed to examine the efficacy and safety of 5 dose levels of once daily administered OFG, compared with once daily administered placebo and once weekly administered dulaglutide. The patients were randomized 5:5:5:5:5:3:3:3:3 to once daily placebo, once weekly dulaglutide+oral placebo or OFG maintenance doses of 3 mg, 12 mg, 24 mg, 36 mg (36-1, 36-2) or 45 mg (45-1, 45-2) treatment groups. The 36 and 45 mg cohorts were subdivided in a 36 mg-1 and 36 mg-2 as well as 45 mg-1 and 45 mg-2 respectively, as different starting doses, dose escalations and dose escalation steps were investigated in these subgroup cohorts (FIG. 4).

### Participants

Eligible male or female participants were >18 years of age with T2D with a HbA1c 7.0% to 10.5% and a body mass index (BMI) of ≥23 kg/m2, treated with diet and exercise alone or with a stable dose of metformin and with a stable body weight (≤5% body weight gain and/or loss) for at least 3 months prior to screening/Visit 1.

### Procedures

The study period included a 2-week screening/lead in period and a 26-week treatment period. During the treatment period, doses were escalated for all treatment groups. The dose escalation period ranged from 4 to 12 weeks depending on dose group. The initial dose was 2 or 3 mg followed by additional escalation steps depending on assigned cohort (FIG. 4). All participants took one capsule orally each day (OFG or matching placebo) and one subcutaneous injection each week (dulaglutide 1.5 mg or matching placebo). All participants were provided healthy eating and exercise education by study personnel throughout the trial.

### Endpoint

The primary endpoint was change in HbA1c from baseline at week 26, comparing OFG doses versus placebo. Secondary endpoints included percentage of participants with HbA1c≤6.5% and of <7.0% at week 26, change from baseline in fasting blood glucose at week 26, change from baseline in body weight, and the effect of OFG versus dulaglutide on change from baseline in HbA1c at week 26. Key Safety and tolerability endpoints included the frequency of patient- and investigator-reported adverse events, rate and incidence of hypoglycaemia events (glucose <70 mg/dL [3.9 mmol/L] and ≥54 mg/dL [3.0 mmol/L], or glucose <54 mg/dL [3.0 mmol/L]), and change in safety laboratory parameters, electrocardiogram, and vital signs.

### Statistical Analysis

A sample size of 370 participants was calculated to provide at least 90% power for testing superiority of OFG versus placebo in the primary endpoint. The estimand used for efficacy analyses was an “efficacy estimand”, representing the average treatment effect for the randomized population if the treatment was administered as intended, thus including data from all randomized participants who received at least one dose of study drug and excluding data after permanent discontinuation of study drug. All tests of treatment effects were conducted at a 2-sided alpha level of 0.05 without adjustment for multiple comparisons, and 2-sided 95% CIs were calculated. To test efficacy of OFG with adequate statistical power, for 36 mg and 45 mg the evaluation of the primary endpoint was made by pooling 2 dose escalation regimens (combining 36 mg-1 and 36 mg-2, for 36 mg, and combining 45 mg-1 and 45 mg-2, for 45 mg).

Safety analyses were conducted by comparing safety of OFG with placebo and with dulaglutide irrespective of adherence to study drug. The safety analyses were conducted using the safety analysis set, which includes all randomized participants who received at least one dose of study drug. The results were presented in summary statistics, point estimates, and 95% confidence intervals along with p-values for treatment comparisons.

### Result

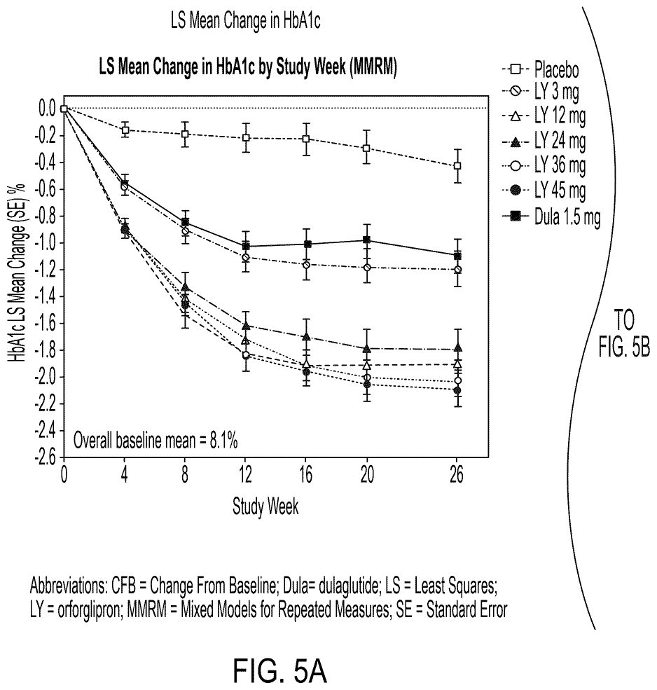

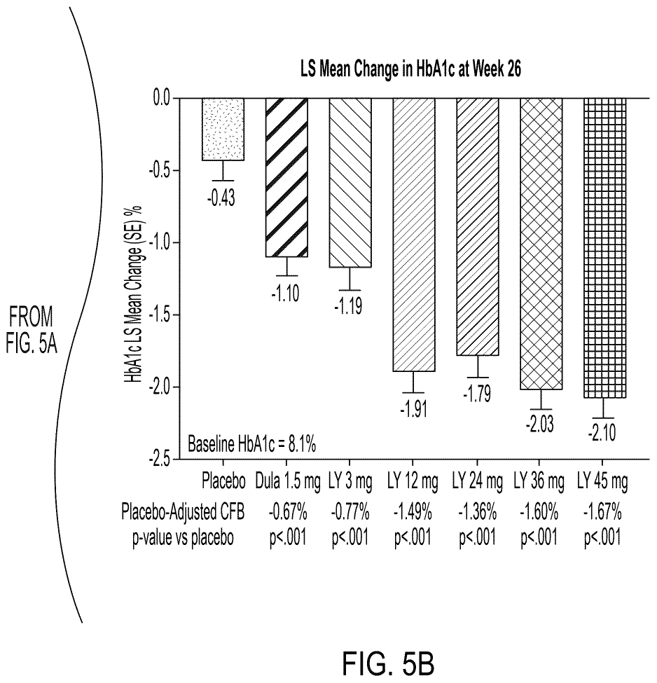

At baseline the mean HbA1c was 8.1%. The mean change in HbA1c from baseline with OFG treatment ranged from −0.77% to −1.67% at week 26, in a dose dependent manner, compared with −0.43% with placebo and −1.1% with dulaglutide 1.5 mg, respectively. The percentage of patients who attained an HbA1c of 7% or less at 26 weeks ranged from 65.2% to 95.8%, versus 24.3% with placebo and 64.1% with dulaglutide 1.5 mg. Treatment with OFG resulted in significant improvements versus placebo in fasting glucose. The most common adverse events with OFG were gastrointestinal, mild to moderate in severity, and occurred primarily during dose escalation. The HbA1c results are shown in FIG. 5.

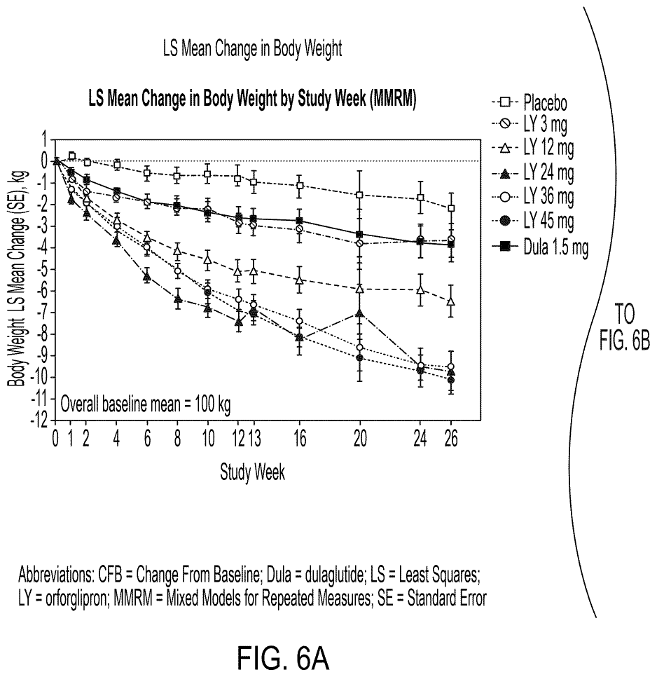

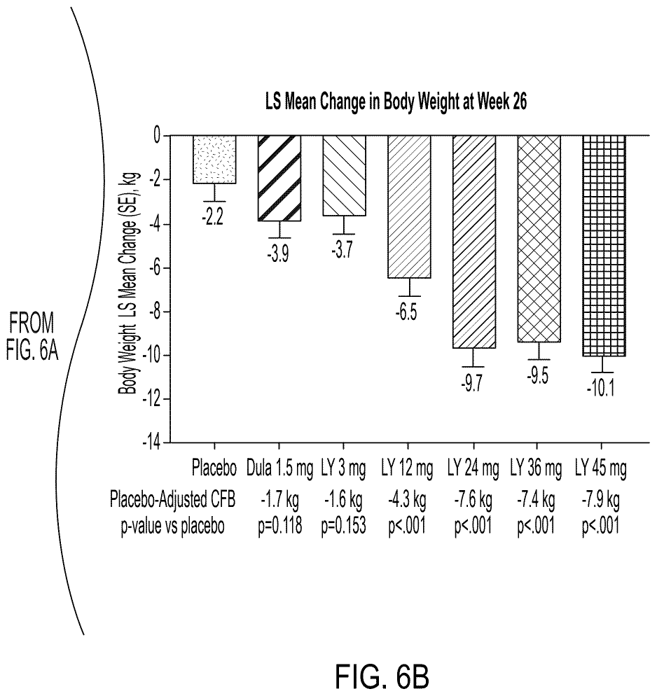

At baseline, the mean body weight was 100.3 kg (standard deviation 21.5). At week 26, mean changes from baseline in body weight for participants who received OFG were −3.7 kg (0.79) for 3 mg, −6.5 kg (0.76) for 12 mg, −9.7 kg (0.85) for 24 mg, −9.5 kg (0.73) for 36 mg, and −10.1 kg (0.71) for 45 mg versus −3.9 kg (0.76) for dulaglutide and −2.2 kg (0.74) for placebo (FIG. 6). OFG was superior to placebo (ETD −4.3 kg [95% CI −6.4 to −2.2] with 12 mg, 7.6 kg [−9.8 to −5.3] with 24 mg, −7.4 kg [−9.4 to −5.3] with 36 mg, −7.9 kg [−9.9 to −5.9] with 45 mg, p<0.0001 OFG 12 mg or greater and superior to dulaglutide (ETD −2.6 kg [−4.7 to −0.5] with 12 mg, −5.9 kg [−8.1 to −3.6] with 24 mg, −5.7 kg [−7.8 to −3.6] with 36 mg, −6.2 kg [−8.3 to −4.2] with 45 mg; p=0.0153 for OFG 12 mg, p<0.0001 for OFG 24 mg, 36 mg, and 45 mg) in decreasing body weight.

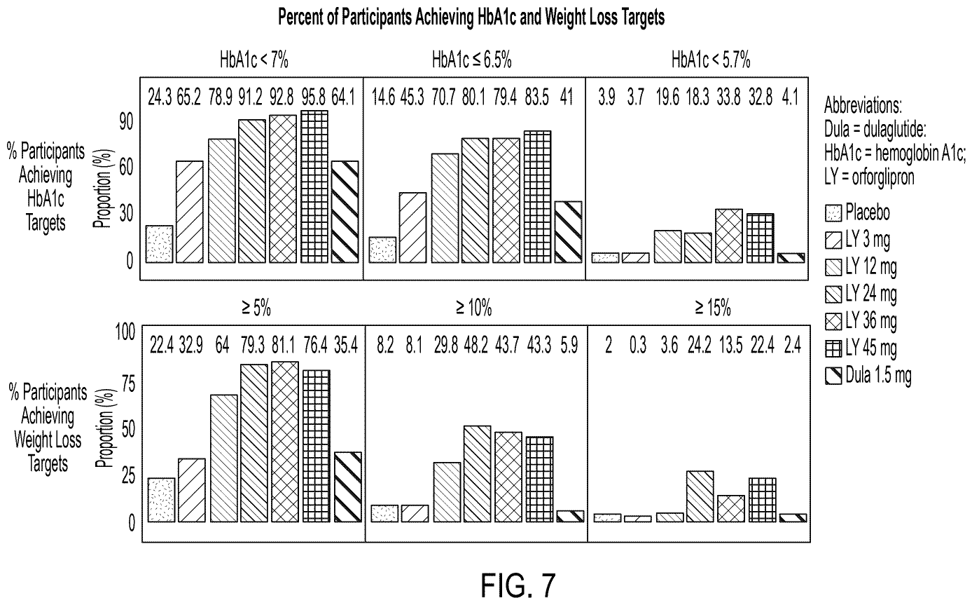

The percentages of participants achieving HbA1c and weight loss targets in the various groups are shown in FIG. 7.

### Additional Treatment Outcomes

At week 26, no statistically significant differences were noted in systolic or diastolic blood pressure compared with dulaglutide or placebo. An increase in mean pulse rate was reported in all OFG treatment arms, with the maximal change occurring around Week 12. At Week 26, participants who received OFG had changes from baseline of +3.0 to +6.1 beats per minute (bpm) compared to +2.3 and −1.6 bpm with dulaglutide and placebo, respectively. More detailed vital sign parameters are provided in the following table.

TABLE 13
Summary and Analysis of Changes from Baseline in Vital Sign Parameters
(Blood Pressure and Pulse Rate) at Baseline and Week 26

| | Baseline | | Week 26 | | | |
|---|---|---|---|---|---|---|
| | | Actual | | Actual | | Within |
| | | Value | | Value | CFB | treatment |
| | | LSM | | LSM | LSM | group p- |
| Treatment | N | (SE) | N | (SE) | (SE)^{c} | value |
| Systolic blood pressure (mm Hg) | | | | | | |
| Placebo | 55 | 135.2 | 51 | 128.9 | −5.5 | .001 |
| | | (1.76) | | (1.65) | (1.65) | |
| OFG 3 mg | 51 | 132.5 | 47 | 126.2 | −8.2 | <.001 |
| | | (1.83) | | (1.72) | (1.72) | |
| OFG 12 mg | 54 | 134.8 | 50 | 126.7 | −7.6 | <.001 |
| | | (1.78) | | (1.66) | (1.66) | |
| OFG 24 mg | 47 | 129.7 | 43 | 127.7 | −6.7 | <.001 |
| | | (1.90) | | (1.80) | (1.80) | |
| OFG 36 mg^{a} | 60 | 133.0 | 56 | 127.1 | −7.3 | <.001 |
| | | (1.70) | | (1.58) | (1.58) | |
| OFG 45 mg^{a} | 63 | 136.7 | 60 | 125.7 | −8.7 | <.001 |
| | | (1.64) | | (1.53) | (1.53) | |
| Dula 1.5 mg | 50 | 135.7 | 49 | 126.5 | −7.9 | <.001 |
| | | (1.84) | | (1.69) | (1.69) | |
| Diastolic blood pressure (mm Hg) | | | | | | |
| Placebo | 55 | 81.5 | 51 | 78.5 | −1.8 | .073 |
| | | (1.11) | | (0.98) | (0.98) | |
| OFG 3 mg | 51 | 78.3 | 47 | 78.3 | −1.9 | .059 |
| | | (1.15) | | (1.02) | (1.02) | |
| OFG 12 mg | 54 | 80.1 | 50 | 79.2 | −1.1 | .262 |
| | | (1.12) | | (0.99) | (0.99) | |
| OFG 24 mg | 47 | 78.4 | 43 | 78.0 | −2.3 | .033 |
| | | (1.20) | | (1.07) | (1.07) | |
| OFG 36 mg^{b} | 60 | 80.6 | 56 | 78.8 | −1.5 | .123 |
| | | (1.07) | | (0.94) | (0.94) | |
| OFG 45 mg^{b} | 63 | 80.6 | 60 | 78.7 | −1.6 | .088 |
| | | (1.04) | | (0.91) | (0.91) | |
| Dula 1.5 mg | 50 | 81.2 | 49 | 77.8 | −2.5 | .016 |
| | | (1.16) | | (1.01) | (1.01) | |
| Pulse Rate (bpm) | | | | | | |
| Placebo | 55 | 72.4 | 51 | 72.0 | −1.6 | .184 |
| | | (1.37) | | (1.19) | (1.19) | |
| OFG 3 mg | 51 | 73.6 | 47 | 76.6 | 3.0 | .016 |
| | | (1.43) | | (1.24) | (1.24) | |
| OFG 12 mg | 54 | 72.4 | 50 | 79.7 | 6.1 | <.001 |
| | | (1.39) | | (1.20) | (1.20) | |
| OFG 24 mg | 47 | 72.2 | 43 | 77.4 | 3.8 | .004 |
| | | (1.49) | | (1.29) | (1.29) | |
| OFG 36 mg^{b} | 60 | 75.6 | 56 | 79.7 | 6.1 | <.001 |
| | | (1.33) | | (1.14) | (1.14) | |
| OFG 45 mg^{b} | 63 | 76.1 | 60 | 79.2 | 5.6 | <.001 |
| | | (1.28) | | (1.10) | (1.10) | |
| Dula 1.5 mg | 50 | 72.1 | 49 | 75.9 | 2.3 | .055 |
| | | (1.44) | | (1.22) | (1.22) | |

Abbreviations: CFB = change from baseline; Dula = dulaglutide; HbA1c = hemoglobin A1c; LSM = least squares mean; MMRM = mixed model repeated measures; N = number of participants in the specified category; SE = standard error.
^{a}Safety analysis - all data available are included for safety analyses, including those from both the treatment period and safety follow-up.
^{b}OFG 36 mg−1 and OFG 36 mg−2 were combined for OFG 36 mg; OFG 45 mg−1 and OFG 45 mg−2 were combined for OFG 45 mg.
^{c}MMRM model for postbaseline measures: log (Actual Measurement/Baseline) = log (Baseline) + Country + Baseline HbA1c Group (≤8.0%, >8.0%) + Treatment + Time + Treatment × Time (Type III sum of squares). Variance-Covariance structure (Actual Measurement) = Unstructured.
Note 1:
Only participants with nonmissing baseline value and at least 1 nonmissing postbaseline value of the response variable were included in analysis.
Note 2:
Model estimates for pooled treatments are estimated via a linear contrast that averages estimates from individual treatment groups. Summary statistics for pooled treatments are calculated based on observed data without linear contrasts.

### Tolerability and Safety

Gastrointestinal adverse events occurred more frequently with participants receiving high initial doses or rapid dose escalation, for example in the OFG 36 mg treatment group, 70.4% of participants in the 36 mg-1 treatment group experienced a gastrointestinal adverse event, versus 44.1% in the 36 mg-2 treatment group.

TABLE 14
Summary of Prevalence, Incidence, and Onset of Nausea, Vomiting, Diarrhea,
and Constipation by Time Interval by Treatment Group over Time Safety
Analysis Set (Note: “LY” in the table below is the same as “OFG”)

| | | LY 3 | LY 12 | LY 24 | LY 36 | LY 36 | LY 45 | LY 45 | Dula |
|---|---|---|---|---|---|---|---|---|---|
| | Placebo | mg | mg | mg | mg−1 | mg−2 | mg−1 | mg−2 | 1.5 mg |
| Time point | N = 55 | N = 51 | N = 56 | N = 47 | N = 27 | N = 34 | N = 31 | N = 32 | N = 50 |
| Nausea | | | | | | | | | |
| Prevalence (%) | | | | | | | | | |
| After the 1st dose | 5.5 | 23.5 | 37.5 | 34.0 | 37.0 | 26.5 | 29.0 | 25.0 | 18.0 |
| 0 ≤ Weeks ≤ 12 | 5.5 | 23.5 | 37.5 | 34.0 | 37.0 | 23.5 | 29.0 | 25.0 | 18.0 |
| 12 < Weeks ≤ 16 | 0 | 6.0 | 5.8 | 13.3 | 15.4 | 11.8 | 10.0 | 9.4 | 4.0 |
| 16 < Weeks ≤ 20 | 0 | 4.2 | 3.8 | 6.7 | 15.4 | 8.8 | 6.7 | 6.5 | 4.1 |
| 20 < Weeks ≤ 26 | 0 | 6.4 | 3.8 | 4.4 | 15.4 | 9.1 | 6.7 | 6.5 | 4.1 |
| 26 < Weeks ≤ End | 0 | 2.9 | 0 | 2.6 | 9.1 | 11.5 | 9.1 | 8.7 | 2.3 |
| of treatment period | | | | | | | | | |
| Incidence (%) | | | | | | | | | |
| After the 1st dose | 5.5 | 23.5 | 37.5 | 34.0 | 37.0 | 26.5 | 29.0 | 25.0 | 18.0 |
| 0 ≤ Weeks ≤ 12 | 5.5 | 23.5 | 37.5 | 34.0 | 37.0 | 23.5 | 29.0 | 25.0 | 18.0 |
| 12 < Weeks ≤16 | 0 | 0 | 0 | 2.2 | 3.8 | 8.8 | 6.7 | 0 | 0 |
| 16 < Weeks ≤ 20 | 0 | 0 | 0 | 0 | 3.8 | 0 | 3.3 | 0 | 0 |
| 20 < Weeks ≤ 26 | 0 | 2.1 | 0 | 0 | 0 | 0 | 0 | 0 | 0 |
| 26 < Weeks ≤ End | 0 | 0 | 0 | 0 | 0 | 0 | 0 | 0 | 0 |
| of treatment period | | | | | | | | | |
| Onset (%) | | | | | | | | | |
| After the 1st dose | 5.5 | 23.5 | 37.5 | 34.0 | 37.0 | 26.5 | 29.0 | 25.0 | 18.0 |
| 0 ≤ Weeks ≤ 12 | 5.5 | 23.5 | 37.5 | 34.0 | 37.0 | 23.5 | 29.0 | 25.0 | 18.0 |
| 12 < Weeks ≤ 16 | 0 | 0 | 0 | 0 | 0 | 2.9 | 0 | 0 | 0 |
| 16 < Weeks ≤ 20 | 0 | 0 | 0 | 0 | 0 | 0 | 0 | 0 | 0 |
| 20 < Weeks ≤ 26 | 0 | 0 | 0 | 0 | 0 | 0 | 0 | 0 | 0 |
| 26 < Weeks ≤ End | 0 | 0 | 0 | 0 | 0 | 0 | 0 | 0 | 0 |
| of period | | | | | | | | | |
| Vomiting | | | | | | | | | |
| Prevalence (%) | | | | | | | | | |
| After the 1st dose | 1.8 | 5.9 | 21.4 | 27.7 | 33.3 | 20.6 | 35.5 | 21.9 | 8.0 |
| 0 ≤ Weeks ≤ 12 | 0 | 5.9 | 19.6 | 25.5 | 33.3 | 17.6 | 35.5 | 21.9 | 6.0 |
| 12 < Weeks ≤ 16 | 0 | 0 | 1.9 | 6.7 | 7.7 | 5.9 | 0 | 3.1 | 0 |
| 16 < Weeks ≤ 20 | 1.9 | 0 | 1.9 | 2.2 | 7.7 | 0 | 0 | 0 | 0 |
| 20 < Weeks ≤ 26 | 0 | 0 | 1.9 | 0 | 7.7 | 0 | 3.3 | 0 | 2.0 |
| 26 < Weeks ≤ End | 0 | 0 | 5.6 | 0 | 4.5 | 0 | 0 | 0 | 0 |
| of treatment period | | | | | | | | | |
| Incidence (%) | | | | | | | | | |
| After the 1st dose | 1.8 | 5.9 | 21.4 | 27.7 | 33.3 | 20.6 | 35.5 | 21.9 | 8.0 |
| 0 ≤ Weeks ≤ 12 | 0 | 5.9 | 19.6 | 25.5 | 33.3 | 17.6 | 35.5 | 21.9 | 6.0 |
| 12 < Weeks ≤ 16 | 0 | 0 | 0 | 4.4 | 3.8 | 5.9 | 0 | 3.1 | 0 |
| 16 < Weeks ≤ 20 | 1.9 | 0 | 0 | 0 | 3.8 | 0 | 0 | 0 | 0 |
| 20 < Weeks ≤ 26 | 0 | 0 | 0 | 0 | 3.8 | 0 | 3.3 | 0 | 2.0 |
| 26 < Weeks ≤ End | 0 | 0 | 2.8 | 0 | 0 | 0 | 0 | 0 | 0 |
| of treatment period | | | | | | | | | |
| Onset (%) | | | | | | | | | |
| After the 1st dose | 1.8 | 5.9 | 21.4 | 27.7 | 33.3 | 20.6 | 35.5 | 21.9 | 8.0 |
| 0 ≤ Weeks ≤ 12 | 0 | 5.9 | 19.6 | 25.5 | 33.3 | 17.6 | 35.5 | 21.9 | 6.0 |
| 12 < Weeks ≤ 16 | 0 | 0 | 0 | 2.2 | 0 | 2.9 | 0 | 0 | 0 |
| 16 < Weeks ≤ 20 | 1.9 | 0 | 0 | 0 | 0 | 0 | 0 | 0 | 0 |
| 20 < Weeks ≤ 26 | 0 | 0 | 0 | 0 | 0 | 0 | 0 | 0 | 2.0 |
| 26 < Weeks ≤ End | 0 | 0 | 2.8 | 0 | 0 | 0 | 0 | 0 | 0 |
| of treatment period | | | | | | | | | |
| Diarrhea | | | | | | | | | |
| Prevalence (%) | | | | | | | | | |
| After the 1st dose | 7.3 | 21.6 | 16.1 | 14.9 | 25.9 | 5.9 | 29.0 | 28.1 | 12.0 |
| 0 ≤ Weeks ≤ 12 | 5.5 | 19.6 | 14.3 | 12.8 | 18.5 | 2.9 | 29.0 | 25.0 | 8.0 |
| 12 < Weeks ≤ 16 | 1.9 | 4.0 | 3.8 | 4.4 | 11.5 | 2.9 | 3.3 | 9.4 | 6.0 |
| 16 < Weeks ≤ 20 | 1.9 | 4.2 | 1.9 | 2.2 | 3.8 | 0 | 3.3 | 3.2 | 6.1 |
| 20 < Weeks ≤ 26 | 0 | 4.3 | 1.9 | 0 | 7.7 | 0 | 3.3 | 3.2 | 4.1 |
| 26 < Weeks ≤ End | 0 | 0 | 5.6 | 0 | 4.5 | 0 | 4.5 | 4.3 | 4.7 |
| of treatment period | | | | | | | | | |
| Incidence (%) | | | | | | | | | |
| After the 1st dose | 7.3 | 21.6 | 16.1 | 14.9 | 25.9 | 5.9 | 29.0 | 28.1 | 12.0 |
| 0 ≤ Weeks ≤ 12 | 5.5 | 19.6 | 14.3 | 12.8 | 18.5 | 2.9 | 29.0 | 25.0 | 8.0 |
| 12 < Weeks ≤ 16 | 0 | 2.0 | 0 | 2.2 | 7.7 | 2.9 | 0 | 6.3 | 4.0 |
| 16 < Weeks ≤ 20 | 1.9 | 2.1 | 0 | 2.2 | 0 | 0 | 0 | 0 | 2.0 |
| 20 < Weeks ≤ 26 | 0 | 0 | 0 | 0 | 3.8 | 0 | 0 | 0 | 0 |
| 26 < Weeks ≤ End | 0 | 0 | 2.8 | 0 | 0 | 0 | 0 | 0 | 0 |
| of treatment period | | | | | | | | | |
| Onset (%) | | | | | | | | | |
| After the 1st dose | 7.3 | 21.6 | 16.1 | 14.9 | 25.9 | 5.9 | 29.0 | 28.1 | 12.0 |
| 0 ≤ Weeks ≤ 12 | 5.5 | 19.6 | 14.3 | 12.8 | 18.5 | 2.9 | 29.0 | 25.0 | 8.0 |
| 12 < Weeks ≤ 16 | 0 | 2.0 | 0 | 0 | 3.8 | 2.9 | 0 | 3.1 | 2.0 |
| 16 < Weeks ≤ 20 | 1.9 | 0 | 0 | 2.2 | 0 | 0 | 0 | 0 | 2.0 |
| 20 < Weeks ≤ 26 | 0 | 0 | 0 | 0 | 3.8 | 0 | 0 | 0 | 0 |
| 26 < Weeks ≤ End | 0 | 0 | 2.8 | 0 | 0 | 0 | 0 | 0 | 0 |
| of treatment period | | | | | | | | | |
| Constipation | | | | | | | | | |
| Prevalence (%) | | | | | | | | | |
| After the 1st dose | 1.8 | 13.7 | 12.5 | 12.8 | 22.2 | 2.9 | 3.2 | 12.5 | 0 |
| 0 ≤ Weeks ≤ 12 | 1.8 | 11.8 | 10.7 | 12.8 | 18.5 | 2.9 | 0 | 12.5 | 0 |
| 12 < Weeks ≤ 16 | 0 | 8.0 | 3.8 | 0 | 11.5 | 2.9 | 0 | 3.1 | 0 |
| 16 < Weeks ≤ 20 | 0 | 2.1 | 1.9 | 2.2 | 11.5 | 2.9 | 0 | 3.2 | 0 |
| 20 < Weeks ≤ 26 | 0 | 2.1 | 3.8 | 0 | 7.7 | 3.0 | 3.3 | 3.2 | 0 |
| 26 < Weeks ≤ End | 0 | 2.9 | 5.6 | 0 | 0 | 3.8 | 4.5 | 0 | 0 |
| of treatment period | | | | | | | | | |
| Incidence (%) | | | | | | | | | |
| After the 1st dose | 1.8 | 13.7 | 12.5 | 12.8 | 22.2 | 2.9 | 3.2 | 12.5 | 0 |
| 0 ≤ Weeks ≤ 12 | 1.8 | 11.8 | 10.7 | 12.8 | 18.5 | 2.9 | 0 | 12.5 | 0 |
| 12 < Weeks ≤ 16 | 0 | 2.0 | 1.9 | 0 | 0 | 0 | 0 | 0 | 0 |
| 16 < Weeks ≤ 20 | 0 | 0 | 0 | 2.2 | 3.8 | 0 | 0 | 0 | 0 |
| 20 < Weeks ≤ 26 | 0 | 0 | 1.9 | 0 | 0 | 0 | 3.3 | 3.2 | 0 |
| 26 < Weeks ≤ End | 0 | 0 | 0 | 0 | 0 | 0 | 0 | 0 | 0 |
| of treatment period | | | | | | | | | |
| Onset (%) | | | | | | | | | |
| After the 1st dose | 1.8 | 13.7 | 12.5 | 12.8 | 22.2 | 2.9 | 3.2 | 12.5 | 0 |
| 0 ≤ Weeks ≤ 12 | 1.8 | 11.8 | 10.7 | 12.8 | 18.5 | 2.9 | 0 | 12.5 | 0 |
| 12 < Weeks ≤ 16 | 0 | 2.0 | 0 | 0 | 0 | 0 | 0 | 0 | 0 |
| 16 < Weeks ≤ 20 | 0 | 0 | 0 | 0 | 3.8 | 0 | 0 | 0 | 0 |
| 20 < Weeks ≤ 26 | 0 | 0 | 1.9 | 0 | 0 | 0 | 3.3 | 0 | 0 |
| 26 < Weeks ≤ End | 0 | 0 | 0 | 0 | 0 | 0 | 0 | 0 | 0 |
| of treatment period | | | | | | | | | |

Abbreviations: Dula = dulaglutide; N = number of participants in a specified treatment group.
Note:
Prevalence refers to the proportion of participants who have an event during a time interval. Incidence refers to the proportion of participants who have a new event during a time interval. Onset refers to the time interval where participants who have an event were first present. After the 1st dose refers to the interval from Week 0 to end of treatment period.

### Example 5—Phase 3 Clinical Study

In this phase 3 clinical study, OFG (Compound 1a hemicalcium salt) is administered to a subject per the Clinical Study Protocol as described herein to compare the effect of OFG with insulin glargine on the incidence of MACE-4 in individuals with T2D, obesity or overweight with at least one weight related comorbidity who are at increased risk for cardiovascular (CV) events. A brief summary of this study is provided in the following table.

| | | | Primary and | | Total | Treatments/ |
|---|---|---|---|---|---|---|
| Ex. No. | | Primary | Key Secondary | | number of | maintenance |
| Study ID | Description | Objective | Endpoints | | participants | dose |
| Ex. 5 | A phase 3, | To | MACE-4: | Event | 2620 | OFG 36 |
| ACHIEVE- | open-label | demonstrate | MI | driven | | mg or |
| 4 (GZGS) | study of once | that OFG is | Stroke | (~122 | | MTD |
| | daily OFG | non- inferior | Hospitalization | MACE- | | Insulin |
| | compared | to insulin | for UA | 4) | | Glargine |
| | with insulin | glargine | CV death | | | 100 U/mL |
| | glargine in | (with a NIM | | | | |
| | adult | of 1.8) in | | | | |
| | participants | occurrence | | | | |
| | with T2D and | of MACE-4 | | | | |
| | obesity or | | | | | |
| | overweight at | | | | | |
| | increased | | | | | |
| | cardiovascular | | | | | |
| | risk | | | | | |

A detailed description of this study is provided below.

TABLE 15

| | |
|---|---|
| Objectives, Endpoints, | |
| and Estimands: Objectives | Endpoints |

Primary

| | |
|---|---|
| To demonstrate that OFG is non- | Time to first occurrence |
| inferior to insulin glargine | of any MACE-4 event |
| (with a non-inferiority margin | MI |
| of 1.8) in occurrence of | stroke |
| MACE-4 events | hospitalization for |
| | unstable angina, or |
| | CV death |

TABLE 16
Intervention Groups and Duration:

| This table lists the | | |
|---|---|---|
| interventions used | | |
| in this clinical study. | | Insulin |
| Intervention Name | OFG | glargine |
| Dosage Level(s) | 1 mg, 3 mg, 6 mg, | prefilled pen containing |
| | 12 mg, 24 mg, and | 300 units (3 mL), |
| | 36 mg capsules | individualized dosing |
| | | to achieve a FBG target |
| | | of ≤100 mg/dl |
| | | (≤5.6 mmol/L) |
| Route of | Oral QD | Subcutaneous |
| Administration | | injection QD |

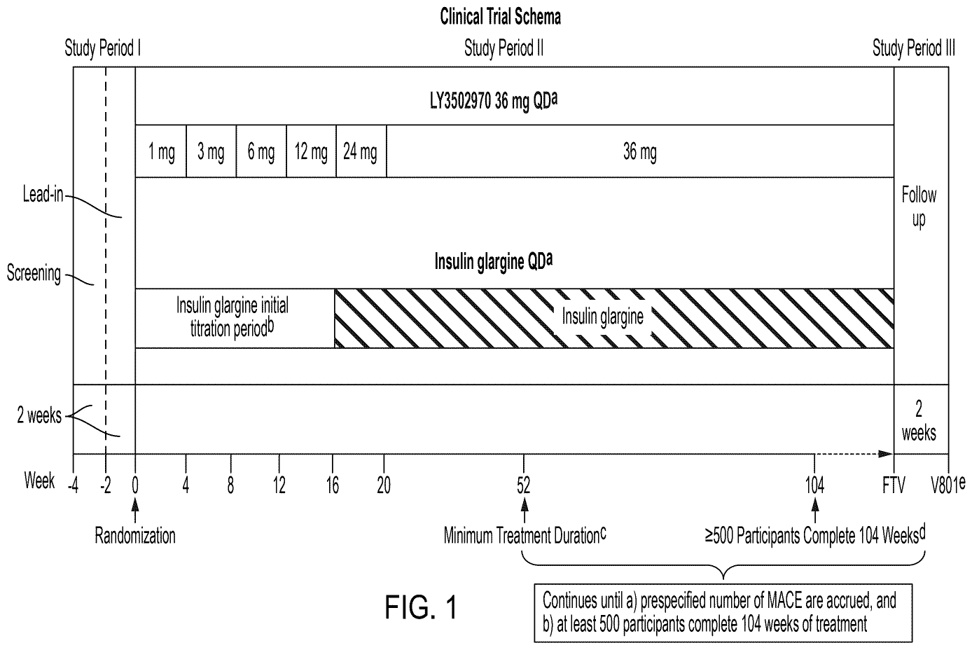

A schema showing patient visits and treatment schedules including time, durations and doses is shown in FIG. 1.

The abbreviations and footnotes used in FIG. 1 are provided below.
FTV=final treatment visit;
MACE=major adverse cardiovascular events;
QD=once daily.
a) All participants will be on stable treatment of at least 1 and no more than 3 oral antihyperglycemic drugs (metformin, sodium-glucose co-transporter 2-inhibitors, or sulfonylurea).
b) The starting dose of insulin glargine will be 10 IU/day, titrated to a FBG≤100 mg/dL (≤5.6 mmol/L) following an insulin dosing schedule. Participants will titrate insulin glargine dose in a weekly manner and will make the dose decision with the investigator for the first 8 weeks (phone or clinic visit). From Week 8 to Week 16 participants will continue weekly titration and will have phone or clinic visit every other week checking the accurate dose decision based on the algorithm.
c) Participants will be on study intervention for at least 12 months and will continue until study completion criteria are met.
d) Participants reaching 104 weeks of treatment prior to study closure will continue repeating visits every 12 weeks until study completion criteria are met.
e) All participants should perform a Visit 801, 2 weeks after their FTV.

### Study Population—Inclusion Criteria

Type of participant and disease characteristics
1. Have a clinical diagnosis of T2D based on the World Health Organization classification or other locally applicable diagnostic standards.
2. Have HbA1c value at screening Visit 1
   a. between ≥7.0% (53 mmol/mol) and ≤10.5% (91 mmol/mol) if background diabetes medication does not include a sulfonylurea, or
   b. between ≥7.5% (58 mmol/mol) and ≤10.5% (91 mmol/mol) if background diabetes medication includes a sulfonylurea.
3. Are on stable treatment of at least 1 and no more than 3 oral antihyperglycemic drugs for at least 90 days before screening Visit 1.
Allowed antihyperglycemic drugs
   metformin (required dose ≥1500 mg/day unless lower dose required per country-specific labeling based on eGFR or other renal function measure),
   SGLT-2 inhibitors, or
   sulfonylurea.
4. Have increased risk of CV events

### Study Intervention(s) and Concomitant Therapy

This table lists the interventions used in this clinical study.

TABLE 17

| Intervention Name | OFG | Insulin glargine |
|---|---|---|
| Dosage Level(s) | 1 mg, 3 mg, 6 mg, | prefilled pen containing |
| | 12 mg, 24 mg, and | 300 units (3 mL), |
| | 36 mg capsules | individualized dosing to |
| | | achieve a FBG target of |
| | | ≤100 mg/dl (≤5.6 mmol/L) |
| Route of | Oral QD | Subcutaneous injection QD |
| Administration | | |

### Participants Randomized to OFG

The starting dose of OFG will be 1 mg QD for 4 weeks, followed by increases in dose every 4 weeks up to 36 mg QD as outlined in this table.

TABLE 18
OFG Dose Escalation

| | | Additional dosing |
|---|---|---|
| Visit (Week) | Weekly Dose | instruction |
| Visit 3 (Week 0) | 1 mg QD | Dose increases occur every |
| Visit 7 (Week 4) | 3 mg QD | 4 weeks until the 36 mg |
| Visit 11 (Week 8) | 6 mg QD | dose, or maximum tolerated |
| Visit 13 (Week 12) | 12 mg QD | dose (MTD) is reached. |
| Visit 15 (Week 16) | 24 mg QD | |
| Visit 16 (Week 20) | 36 mg QD | |

OFG is administered orally once daily.

### Participants Randomized to Insulin Glargine

The starting dose of insulin glargine will be 10 IU/day, participants should administer their daily doses at the time of day agreed upon between the participant and the investigator, typically before bedtime. Insulin glargine will be titrated to a FBG≤100 mg/dL (≤5.6 mmol/L) following the insulin titration schedule in the Treat-to-Target trial (adapted from Riddle et al. 2003).

TABLE 19
Insulin Dose Schedule

| | |
|---|---|
| Median Fasting Blood Glucose^{a} | Dose Adjustment of Insulin |

| mg/dL | mmol/L | Glargine (units/day) |
|---|---|---|
| <70 | <3.9 | Decrease by 2 to 4 units^{b, c} |
| 70 to 99 | 3.9 to 5.5 | No adjustment |
| 100 to 119 | 5.6 to 6.6 | Increase by 2 units |
| 120 to 139 | 6.7 to 7.7 | Increase by 4 units |
| 140 to 179 | 7.8 to 9.9 | Increase by 6 units |
| ≥180 | ≥10.0 | Increase by 8 units |

^{a}Based on the last 3 fasting SMBG values.
^{b}Dose should also be decreased by 2 to 4 units in the following situations
If multiple episodes of non-severe hypoglycemia were recorded during the assessment period at any time during the day; and/or
If at least 1 episode that met the criteria for severe hypoglycemia (events requiring assistance to administer therapy) or was associated with SMBG value <54 mg/dL (<3.0 mmol/L) was recorded during the assessment period.
^{c}If only 1 hypoglycemic episode with SMBG value ≥54 mg/dL (≥3.0 mmol/L) and <70 mg/dL (<3.9 mmol/L) was recorded, insulin dose should not be changed.

### Efficacy Assessments

### Assessments for the Primary Objective

The primary objective of the study is to demonstrate that OFG is non-inferior to insulin glargine with a non-inferiority margin of 1.8 in occurrence of MACE-4 events. The endpoints include CEC-confirmed MI, stroke, hospitalization for unstable angina and CV death.

### Example 6—Phase 1 Clinical Data

In randomized, open-label, crossover Phase 1 clinical studies A and B, the effect of food on OFG concentration in healthy adults after a single 3 mg dose (study A) and after a 16 mg daily dose (study B), which was reached by weekly dose escalation starting with 2 mg, was evaluated.

Based on statistical analysis, Cmax was 23% (study A) and 21% (study B) lower in the fed state. AUC(0-∞), and AUC(0-24) were 24% (study A) and 18% (study B) lower when administered with food. Tmax and T½ were similar with or without food. In study A and Study B, treatment emergent adverse events (TEAEs) were mild. In study B, TEAEs occurred most often with the starting dose and decreased with dose escalation. The most common TEAEs in study B were decreased appetite (69.7%), nausea (48.5%), and vomiting (45.5%).

Overall mean exposure to OFG based on Cmax and AUC was numerically lower when administered with food and had little effect on Tmax and T½. These PK differences between fed and fasted administrations are not expected to result in clinically meaningful differences in OFG safety and efficacy based on exposure response relationship.

TABLE 20

| | | STUDY B -16 mg |
|---|---|---|
| | STUDY A - 3 mg | (with titration) |
| N | 12 | 34 |
| Age (years)^{a} | 45.0 (10.9) | 42.8 (10.2) |
| Weight (kg)^{a} | 82.3 (9.8) | 73.1 (10.4) |

| | Fasted | Fed | Fasted | Fed |
|---|---|---|---|---|
| Cₘₐₓ (ng/mL)^{b} | 13.4 (44%) | 10.1 (44%) | 80.5 (64%) | 67.5 (40%) |
| AUC (ng · h/ml)^{b} | 362 (33%)^{c} | 260 (43%)^{c} | 1200 (58%)^{d} | 1050 (40%)^{d} |
| Tₘₐₓ (h)^{e} | 8.0 (4.0-24.0) | 8.0 (4.0-12.0) | 8.0 (4.0-16.0) | 8.0 (4.0-24.0) |
| T_{1/2} (h)^{f} | 29.5 (19.6-50.6) | 27.9 (19.9-37.9) | 26.0 (8.0-73.4) | 24.6 (8.7-87.1) |

^{a}mean (SD),
^{b}geometric LS mean (geometric CV %),
^{c}AUC_{(0-∞)},
^{d}AUC₍₀₋₂₄₎,
^{e}median (range),
^{f}geometric mean (range)

### Examples 7-12—Additional Phase 3 Clinical Studies

Examples 7-12 describe additional phase 3 clinical studies which are summarized in Table 21 below. OFG (hemicalcium salt of Compound 1a) is administered to a subject per the respective Clinical Study Protocols, the contents of which are incorporated herein by reference, to study the effect of OFG for treating individuals with T2D, obesity, or overweight with at least one weight related comorbidity.

TABLE 21
Summary of Additional Phase 3 Clinical Trials

| | | | | | | |
|---|---|---|---|---|---|---|
| | | | Primary | Treatment | | |
| | | | and Key | duration | Total | Treatments/ |
| Ex. No. | | Primary | Secondary | for primary | number of | Maintenance |
| Study ID | Description | Objective | Endpoints | objective | participants | Doses |

Type 2 Diabetes (T2D)

| | | | | | | |
|---|---|---|---|---|---|---|
| Ex. 7 | A phase 3, | To | HbA1c | 40 weeks | 520 | OFG 3 mg |
| ACHIEVE- | randomized, | demonstrate | % | | | OFG 12 mg |
| 1 (GZGT) | double-blind | that OFG 3 | participants | | | OFG 36 mg |
| | study to | mg, 12 mg, | attaining | | | Placebo |
| | investigate | and/or 36 | HbA1c <7% | | | |
| | the efficacy | mg are | and ≤6.5% | | | |
| | and safety of | superior to | Fasting | | | |
| | once daily | placebo in | glucose | | | |
| | oral OFG | glycemic | Body | | | |
| | compared | control | weight | | | |
| | with placebo | | nonHDL | | | |
| | in adult | | and | | | |
| | participants | | triglycerides | | | |
| | with T2D and | | | | | |
| | inadequate | | | | | |
| | glycemic | | | | | |
| | control with | | | | | |
| | diet and | | | | | |
| | exercise alone | | | | | |
| Ex. 8 | A phase 3, | To | HbA1c | 40 weeks | 888 | OFG 3 mg |
| ACHIEVE- | randomized, | demonstrate | % | | | OFG 12 mg |
| 2 (GZGV) | open-label | that OFG 3 | participants | | | OFG 36 mg |
| | study to | mg, 12 mg | attaining | | | Dapagliflozin |
| | investigate | and/or 36 mg | HbA1c <7% | | | 10 mg |
| | the efficacy | are | and ≤6.5% | | | |
| | and safety of | noninferior | Fasting | | | |
| | once daily | to | glucose | | | |
| | oral OFG | dapagliflozin | Body | | | |
| | compared | in glycemic | weight | | | |
| | with | control, | nonHDL | | | |
| | dapagliflozin | using an | and | | | |
| | in adult | NIM of | triglycerides | | | |
| | participants | 0.3% | Systolic | | | |
| | with type 2 | | blood | | | |
| | diabetes and | | pressure | | | |
| | inadequate | | | | | |
| | glycemic | | | | | |
| | control with | | | | | |
| | metformin | | | | | |
| Ex. 9 | A phase 3, | To | HbA1c | 52 weeks | 1576 | OFG 12 mg |
| ACHIEVE- | randomized, | demonstrate | % | | | OFG 36 mg |
| 3 (GZGU) | open-label | that | participants | | | Oral |
| | study to | (i) OFG 36 | attaining | | | semaglutide |
| | investigate | mg is | HbA1c <7% | | | 7 mg |
| | the efficacy | noninferior | and ≤6.5% | | | Oral |
| | and safety of | to | Fasting | | | semaglutide |
| | once daily | semaglutide | glucose | | | 14 mg |
| | oral OFG | 14 mg in | Body | | | |
| | compared | glycemic | weight | | | |
| | with oral | control | | | | |
| | semaglutide | and/or | | | | |
| | in adult | (ii) OFG 12 | | | | |
| | participants | mg is | | | | |
| | with type 2 | noninferior | | | | |
| | diabetes and | to | | | | |
| | inadequate | semaglutide | | | | |
| | glycemic | 7 mg in | | | | |
| | control with | glycemic | | | | |
| | metformin | control | | | | |
| Ex. 10 | A phase 3, | To | HbA1c | 40 weeks | 520 | OFG 3 mg |
| ACHIEVE- | randomized, | demonstrate | % | Note: | | OFG 12 mg |
| 5 (GZGW) | double-blind | that OFG 12 | participants | During the | | OFG 36 mg |
| | study to | mg and/or | attaining | study, all | | Placebo |
| | investigate | 36 mg is | HbA1c <7% | participants | | |
| | the efficacy | superior to | and ≤6.5% | will be on | | |
| | and safety of | placebo in | Fasting | background | | |
| | once daily | glycemic | glucose | treatment | | |
| | oral OFG | control | Body | with insulin | | |
| | compared | | weight | glargine | | |
| | with placebo | | nonHDL | alone; or a | | |
| | in adult | | and | combination | | |
| | participants | | triglycerides | of insulin | | |
| | with type 2 | | Systolic | glargine | | |
| | diabetes and | | blood | with (i) | | |
| | inadequate | | pressure | metformin, | | |
| | glycemic | | | or (ii) | | |
| | control with | | | SGLT-2 | | |
| | insulin | | | inhibitor, or | | |
| | glargine, with | | | (iii) both | | |
| | or without | | | metformin | | |
| | metformin | | | and SGLT-2 | | |
| | and/or SGLT- | | | inhibitor. | | |
| | 2 inhibitor | | | | | |

Chronic Weight Management

| | | | | | | |
|---|---|---|---|---|---|---|
| Ex. 11 | A Phase 3, | To | Body | 72 weeks | 3000 | OFG 6 mg |
| ATTAIN- | randomized, | demonstrate | weight | | | OFG 12 mg |
| 1 (GZGP) | double-blind | that OFG 6 | % of | | | OFG 36 mg |
| | study to | mg, 12 mg, | participants | | | Placebo |
| | investigate | and/or 36 | who achieve | | | |
| | the efficacy | mg QD is | a body | | | |
| | and safety of | superior to | weight | | | |
| | once-daily | placebo for | reduction of: | | | |
| | oral OFG | body weight | ≥5%, ≥10%, ≥15%, | | | |
| | compared | | or ≥20% | | | |
| | with placebo | | Waist | | | |
| | in adult | | circumference | | | |
| | participants | | nonHDL and | | | |
| | with obesity | | triglycerides | | | |
| | or overweight | | Systolic | | | |
| | with weight- | | blood | | | |
| | related | | pressure | | | |
| | comorbidities | | | | | |
| Ex. 12 | A Phase 3, | To | Body | 72 weeks | 1500 | OFG 6 mg |
| ATTAIN- | randomized, | demonstrate | weight | | | OFG 12 mg |
| 2 (GZGQ) | double-blind | that OFG 6 | % of | | | OFG 36 mg |
| | study to | mg, 12 mg | participants | | | Placebo |
| | investigate | and/or 36 | who achieve | | | |
| | the efficacy | mg is | a body | | | |
| | and safety of | superior to | weight | | | |
| | once-daily | placebo in | reduction of: | | | |
| | oral OFG | change from | ≥5%, ≥10%, ≥15% | | | |
| | compared | baseline for | Waist | | | |
| | with placebo | body weight | circumference | | | |
| | in adult | | HbA1c and | | | |
| | participants | | other | | | |
| | with obesity | | glycemic | | | |
| | or overweight | | control | | | |
| | and type 2 | | measures | | | |
| | diabetes | | nonHDL and | | | |
| | | | triglycerides | | | |
| | | | Systolic | | | |
| | | | blood | | | |
| | | | pressure | | | |

1. A method for treating a subject with type 2 diabetes (T2D), obesity, or overweight with at least one weight related comorbidity in need of such treatment, comprising:
orally administering to the subject a Compound 1 having the following structure: 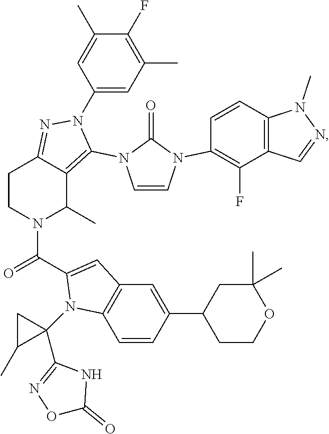 [SMILES: Cc1cc(-n2nc3c(c2-n2ccn(-c4ccc5c(cnn5C)c4F)c2=O)C(C)N(C(=O)c2cc4cc(C5CCOC(C)(C)C5)ccc4n2C2(c4noc(=O)[nH]4)CC2C)CC3)cc(C)c1F]
or a pharmaceutically acceptable salt thereof, or a hydrate of the compound or the pharmaceutically acceptable salt;
at a once daily (QD) dose selected from 1 mg, 2 mg, 3 mg, 6 mg, 12 mg, 24 mg, 36 mg, and a combination thereof, according to a dosing schedule selected from (a)-(d):
(a) starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks;
(b) starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks;
(c) starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks; and
(d) starting at a dose of 1 mg QD, and administering at this dose for 4 weeks; and
then increasing the dose to 3 mg QD, and administering at this dose as a maintenance dose for at least 4 weeks.

2. (canceled)

3. The method of claim 1, wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks.

4. (canceled)

5. The method of claim 1, wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks.

6. The method of claim 1, wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks.

7. The method of claim 1, wherein Compound 1, or a pharmaceutically acceptable salt thereof, or a hydrate of the compound or the pharmaceutically acceptable salt, is administered according to the following dosing schedule:
starting at a dose of 1 mg QD, and administering at this dose for 4 weeks; and
then increasing the dose to 3 mg QD, and administering at this dose as a maintenance dose for at least 4 weeks.

8. The method of claim 1, wherein the doses are administered to the subject for a total treatment time selected from at least 40 weeks, at least 52 weeks, at least 72 weeks, and at least 104 weeks; and
wherein the total treatment time comprises dose escalation period and dose maintenance period.

9. The method of claim 1, wherein the method is for treating a subject with T2D mellitus for improved glycemic control; and
wherein the oral administration of Compound 1, or a pharmaceutically acceptable salt thereof, or a hydrate of the compound or the pharmaceutically acceptable salt, results in a reduction of the subject's HbA1c level of at least 1.0%.

10. The method of claim 9, wherein the reduction of the HbA1c level is at least 1.2%.

11. The method of claim 1, wherein the method is for treating a subject with obesity or overweight with at least one weight related comorbidity; and
wherein the oral administration of Compound 1, or a pharmaceutically acceptable salt thereof, or a hydrate of the compound or the pharmaceutically acceptable salt, results in a weight reduction of at least 5%.

12. The method of claim 11, wherein the weight reduction is at least 10%.

13. The method of claim 11, wherein the weight reduction is at least 5 kg.

14. The method of claim 1, wherein the subject is not restricted from food or water intake within 30 minutes before or after each oral administration of Compound 1, or a pharmaceutically acceptable salt thereof, or a hydrate of the compound or the pharmaceutically acceptable salt.

15. The method of claim 1, wherein oral administration of one or more escalation doses prior to the administration of a maintenance dose results in less treatment related side effects.

16. The method of claim 1, wherein:
oral administration of Compound 1, or a pharmaceutically acceptable salt thereof, or a hydrate of the compound or the pharmaceutically acceptable salt, is co-administered with a second active agent concomitantly or sequentially; and
wherein the second active agent is selected from the group consisting of: an amylin receptor non-peptide agonist, a glucagon receptor non-peptide agonist, a glucose-dependent insulinotropic polypeptide (GIP) non-peptide agonist, a peptide tyrosine-tyrosine (PYY) non-peptide agonist, and a mixture thereof.

17. A method for treating a subject with type 2 diabetes (T2D), obesity, or overweight with at least one weight related comorbidity in need of such treatment, comprising:
orally administering to the subject a Compound 1, or a pharmaceutically acceptable salt thereof, or a hydrate of the compound or the pharmaceutically acceptable salt;
at a once daily (QD) dose selected from 1 mg, 2 mg, 3 mg, 6 mg, 12 mg, 24 mg, 36 mg, and a combination thereof;
wherein the subject is not restricted from food or water intake within 30 minutes before or after the oral administration of Compound 1, or a pharmaceutically acceptable salt thereof, or a hydrate of the compound or the pharmaceutically acceptable salt.

18. The method of claim 17, wherein one or more escalation doses are administered to the subject prior to the administration of a maintenance dose; and
wherein the administration of the escalation doses results in less treatment related side effects as compared to a treatment without an escalation dose.

19. The method of claim 18, wherein the maintenance dose is 36 mg QD, and wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks;
then increasing the dose to 12 mg QD, and administering at this dose for 4 weeks;
then increasing the dose to 24 mg QD, and administering at this dose for 4 weeks; and
then increasing the dose to 36 mg QD, and administering at this dose as a maintenance dose for at least 4 weeks.

20. The method of claim 1, wherein each dose is a free acid equivalent amount of Compound 1.

21. The method of claim 1, wherein Compound 1, or a pharmaceutically acceptable salt thereof, or a hydrate of the compound or the pharmaceutically acceptable salt, is a hemicalcium salt of the following compound or a hydrate of the hemicalcium salt:

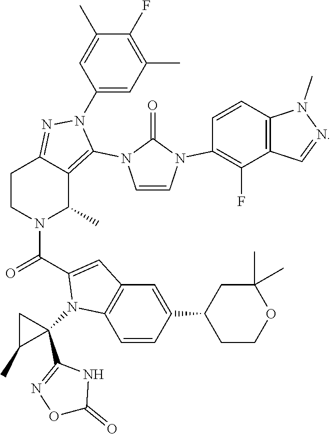 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

22. The method of claim 1, wherein Compound 1, or a pharmaceutically acceptable salt thereof, or a hydrate of the compound or the pharmaceutically acceptable salt, is administered to the subject as an adjunct to diet and exercise.

23-31. (canceled)

32. The method of claim 18, wherein the maintenance dose is 12 mg QD, and wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks;
then increasing the dose to 6 mg QD, and administering at this dose for 4 weeks; and
then increasing the dose to 12 mg QD, and administering at this dose as a maintenance dose for at least 4 weeks.

33. The method of claim 18, wherein the maintenance dose is 6 mg QD, and wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks;
then increasing the dose to 3 mg QD, and administering at this dose for 4 weeks; and
then increasing the dose to 6 mg QD, and administering at this dose as a maintenance dose for at least 4 weeks.

34. The method of claim 18, wherein the maintenance dose is 3 mg QD, and wherein Compound 1, or a pharmaceutically acceptable salt thereof, or a hydrate of the compound or the pharmaceutically acceptable salt, is administered to the subject according to the following dosing schedule:
starting at a dose of 1 mg QD, and administering at this dose for 4 weeks; and
then increasing the dose to 3 mg QD, and administering at this dose as a maintenance dose for at least 4 weeks.

35. The method of claim 1, wherein the administration of the escalation dose prior to the administration of a maintenance dose results in acceptable side effects.
